(19) United States
(12) Patent Application Publication
(10) Pub. No.: US 2009/0326351 A1
(43) Pub. Date: Dec. 31, 2009

(54) Signal Processing Mirroring Technique

(75) Inventors: Paul Stanley Addison, Edinburgh (GB); James Watson, Dunfermline (GB)

Correspondence Address: Nellcor Puritan Bennett LLC;ATTN: IP Legal, 6135 Gunbarrel Avenue, Boulder, CO, 80301, US

(73) Assignee: Nellcor Puritan Bennett Ireland, Mervue (IE)

(21) Appl. No.: 12/437,311

(22) Filed: May 7, 2009

Related U.S. Application Data
(60) Provisional application No. 61/077,062, filed on Jun. 30, 2008.
(60) Provisional application No. 61/077,130, filed on Jun. 30, 2008.

(51) Int. Cl. A61B 5/02; A61B 5/1455

(52) U.S. Cl. 600/324; 600/500

(57) ABSTRACT

Embodiments may include systems and methods capable of processing an original signal by selecting and mirroring portions of the signal to create a new signal for further analysis. In an embodiment, the signal may be a photoplethysmograph (PPG) signal and the new signal may be further analyzed using continuous wavelet transforms. Any suitable number of reconstructed new signals may be created from the original signal and scalograms may be derived at least in part from the new signals. Ridges may be extracted from the scalograms of the new signals and secondary scalograms may be further derived from the ridges. A sum along amplitudes technique may be applied to a selected scalogram and may be plotted as a function of the scale of the scalogram. Desired information, such as respiration information within the original signal, may be identified from the plot.

### CROSS-REFERENCE TO RELATED APPLICATIONS

This application claims the benefit of U.S. Provisional Application No. 61/077,062, filed Jun. 30, 2008 and U.S. Application No. 61/077,130, filed Jun. 30, 2008, which are hereby incorporated by reference herein in their entireties.

### SUMMARY

The present disclosure relates to signal processing systems and methods, and more particularly, to systems and methods for processing an original signal by selecting and mirroring one or more portions of the original signal to create a new signal for further analysis.

In an embodiment, a signal may be selected and mirrored to create a new signal for further analysis. The signal may be from any suitable source and may contain one or more repetitive components. In an embodiment, the selected signal is a portion of the original signal. The portion may be selected using any suitable method based on its characteristics, or characteristics of the original signal (e.g., using local maximum and minimum values, or using second derivatives to find one or more turning points, of the original signal). By selecting a portion of the original signal and mirroring that portion, undesirable artifacts caused by the non-selected portion of the signal during further analysis may be removed and other benefits may be achieved. In an embodiment, additional portions of the original signal may be selected, mirrored, and added to the new signal. Alternatively, separate new signals may be created from the various mirrored portions.

For purposes of illustration, and not by way of limitation, in an embodiment disclosed herein the original signal is a photoplethysmograph (PPG) signal obtained from any suitable source, such as a pulse oximeter, and selected portions are the up and down stroke of a pulse (a pulse is a portion of the PPG signal corresponding to a heart beat), which are used to create separate new signals for further analysis. Further analysis includes determining respiration rate from the PPG signal using Secondary Wavelet Feature Decoupling (SWFD) applied to the new signals. In an embodiment, mirroring up and down strokes to create separate new signals may result in an improved analysis of the original PPG signal. Using a mirroring algorithm that includes forced symmetry (e.g., mirroring a selected up stroke or down stroke about a desired axis creates a pulse that is symmetrical about that desired axis, and a new signal may be constructed from any suitable number of symmetrical pulses) may be beneficial because, for example, it removes undesired aspects of the original signal and improves the accuracy of the respiration rate determination. Using the mirroring algorithm may also significantly improve the number of samples, or the percentage of patient data, from which a patient's respiration rate may be determined effectively. Using the mirroring algorithm may further improve the standard deviation of the differences observed between computing the respiration rate using the mirroring algorithm and computing the respiration rate using another method (e.g., by counting one or more respiration features in a patient's nasal thermistor signal). A tradeoff to using the mirroring algorithm, however, may include an increase in the amount of invalid data that may not be used to determine the patient's respiration rate. Data may be considered invalid if it is the result of excessive movement by a patient, or excessive changes in the spacing between a patients heart beats or other excessive changes in the patient's heart rate. Data also may be considered invalid if the pulse oximeter probe has fallen off or become detached from the patient, or if the PPG signal is excessively corrupted due to noise.

In an embodiment, multiple up and down strokes are mirrored and combined to create new signals. The new signals are referred to herein as a “reconstructed up signal” for the series of pulses created from mirroring one or more up strokes selected from an original signal, or a “reconstructed down signal” for the series of pulses created from mirroring one or more down strokes selected from the original signal. The reconstruction process (i.e., the process of creating pulses by mirroring a series of selected up or down strokes, and creating a new signal from the pulses) may be performed in real time, using a time window smaller than the entire time window over which the original PPG signal may be collected, or the process may be performed offline, using the entire time window of data over which the PPG signal was collected.

Up and down strokes may be selected using any suitable approach. For example, one or more pulses of the original signal may be selected based upon maximum and minimum values of the signal, or using second derivatives to find one or more turning points of the original signal. In an embodiment, the PPG signal may be filtered using, for example, a bandpass or low pass filter to filter out frequencies higher and lower than the range of typical heart rates. Once a pulse is selected, its up stroke may be separated from its down stroke using any suitable method. For example, the up stroke may be separated from the down stroke at the point where the local maximum perpendicular to the two turning points may intersect the selected pulse.

In an embodiment, the reconstructed up and down signals may be further manipulated prior to further analysis. For example, each pulse of the mirrored signal may be expanded or shortened independently of the other pulses in the mirrored signals. For example, each of the pulses created by mirroring up or down strokes in the PPG embodiment may be stretched or compressed to make the time period for each pulse equal in size, where all of the time periods together equal the time period over which the original signal was collected or is being analyzed. Alternatively, each pulse of the mirrored signal may not be stretched to match a time period, but may instead be stretched or compressed to any desired size based at least in part on another time period or based at least in part on an individual or predetermined number of signal pulses. In an embodiment, for example, each mirrored up pulse may be stretched or compressed to match the size of the up stroke used in the mirroring combined with its corresponding down stroke. The same process may be performed on each mirrored down pulse. In an embodiment, the mirrored pulses may be equally stretched or compressed to match the time period over which the signal was collected or is being analyzed.

The frequency modulation that occurs when one or more of the pulses in the mirrored signals is stretched or compressed may be converted into amplitude modulation by increasing or decreasing the amplitude of each of the pulses in the mirrored signals in relation to the amount of individual stretching or compressing. This may increase the amplitude modulation that may already exist in the mirrored pulses due to, for example, baseline changes in an original PPG signal. Translating the effect of the frequency modulation into amplitude modulation within the mirrored signals may alter the effect of certain components within the original signal on the analysis of the original signal. The amplitude of, for example, the pulses in the mirrored signals may be modulated or augmented to create the reconstructed signals if each of the pulses was stretched or compressed independently of each other. Alternatively, the amplitude of each of the pulses in the mirrored signals may be the same if the frequency modulation applied to the mirrored signal stretched or compressed each pulse individually to create reconstructed signals with uniform amplitude. In an embodiment, the reconstructed signals may include pulses that may vary in amplitude and frequency.

The reconstructed up and down signals may be further analyzed using any suitable method, including for example (and as described below for purposes of illustration), SWFD. In an embodiment of the disclosure, only one reconstructed signal, instead of both reconstructed signals, may be analyzed. A primary up scalogram and a primary down scalogram may be derived at least in part from the reconstructed up signal and down signal using any suitable method. For example, the up scalogram and the down scalogram may be derived using continuous wavelet transforms, including using a mother wavelet of any suitable characteristic frequency or form such as the Morlet wavelet with a particular scaling factor value. The up scalogram and the down scalogram also may be derived over any suitable range of scales. The resultant up scalogram and down scalogram may include ridges corresponding to at least one area of increased energy that may be analyzed further using any suitable method, for example using secondary wavelet feature decoupling.

The up ridge and the down ridge of the up and down scalograms may be extracted using any suitable method. For example, the up ridge and the down ridge may represent that at a particular scale value, the PPG signal may contain high amplitudes corresponding to the characteristic frequency of that scale. By extracting and further analyzing the ridges, information concerning the nature of the signal component associated with the underlying physical process causing a primary band on the up and down scalograms may also be extracted when the primary band itself is, for example, obscured in the presence of noise or other erroneous signal features. Secondary wavelet feature decoupling may be applied to each of the up and down ridges to derive secondary up and down scalograms. The secondary wavelet feature decoupling technique may provide desired information about the primary band by examining the amplitude modulation of a secondary band, such amplitude modulation being based at least in part on the presence of the signal component in the PPG signal that may be related to the primary band. This secondary wavelet decomposition of the up and down ridges allows for information concerning the band of interest to be made available as secondary bands for each of the secondary tip and down scalograms. The secondary up and down scalograms may be derived using wavelets within a range of scales from any suitable minimum value up to any suitable maximum value and may be derived using any suitable scaling factor value for the wavelet.

In an embodiment, secondary scalograms may be derived again at a lower scaling factor value so as to break up false ridges within the first set of secondary scalograms. The ridge fragments formed within the repeated secondary scalograms may be used to identify stable regions within the first set of secondary scalograms. The ridge fragments may be analyzed to select one or more desired ridges using any suitable method. For example, a time window that may vary both in width and in start position (e.g., start time) may be slid across the one or more up repeated scalograms and the one or more down repeated scalograms. The ridge fragments within the time window may be parameterized in terms of a weighting of the standard deviation of the path that the particular ridge fragment may take, in units of scale, the length of the ridge fragment, the proximity of the ridge fragment to other ridge fragments, and/or any other suitable weighting characteristics. The ridge having the highest weighting may be chosen for further processing. In an embodiment, the ridge having the highest weighting may be used to identify and select a stable region within one of the generated scalograms.

A sum along amplitudes technique may be applied to at least a portion of the band corresponding to the selected ridge or at least a portion (e.g., the identified stable region) of the selected secondary scalogram using any suitable method. The technique of applying a sum along amplitudes may be applied to any secondary wavelet feature decoupling method of any suitable original signal. Alternatively, the sum along amplitudes technique may be applied to the entire secondary up scalogram or secondary down scalogram. The sum along amplitudes technique also may be applied to any continuous wavelet transform of any suitable signal, such as a wavelet transform of the original PPG signal. The sum along amplitudes technique may sum the amplitudes (e.g., the energy) for each scale within a range of scales across a time window. In an embodiment, the sum along amplitudes technique may be applied to a scalogram composite, or a superposition formed from the secondary scalograms. The sum along amplitudes function may be plotted as a function of any suitable value, such as scale value. From the plot, the first peak or edge moving from a direction of decreasing scale along the sum of scales may be identified. The first peak or edge may have analytical value in relation to the original signal from which the secondary wavelet transforms were derived.

In an embodiment, a signal processing method is provided. The method may include selecting a first portion of an original signal, mirroring the first portion of the original signal about a first vertical axis to create a mirrored first portion, selecting a subsequent second portion of the original signal, mirroring the second portion of the original signal about a second vertical axis to create a mirrored second portion, combining the mirrored first portion and the mirrored second portion to create a new signal, and analyzing the new signal.

In an embodiment, a system for processing a signal is provided. The system may include an input signal generator for generating the signal. The system may also include a processor coupled to the input signal generator. The processor is configured to select a first portion of an original signal, mirror the first portion of the original signal about a first vertical axis to create a mirrored first portion, select a subsequent second portion of the original signal, mirror the second portion of the original signal about a second vertical axis to create a mirrored second portion, combine the mirrored first portion and the mirrored second portion to create a new signal, and analyze the new signal. The system may also include an output coupled to the processor. The output is configured to display the new signal analyzed by the processor. In an embodiment a signal processing method is provided. The method may include transforming a signal using a wavelet transform, generating a scalogram based at least in part on the transformed signal, selecting a region of the scalogram, summing amplitudes for each scale in the region, identifying a maximum sum, and selecting a desired scale associated with the maximum sum.

In an embodiment, a system for processing a signal is provided. The system may include an input signal generator for generating the signal. The system may also include a processor coupled to the input signal generator. The processor is configured to transform the signal using a wavelet transform, generate a scalogram based at least in part on the transformed signal, select a region of the scalogram, sum amplitudes for each scale in the region, identify a maximum sum, and select a desired scale associated with the maximum sum. The system may also include an output coupled to the processor. The output is configured to display the desired scale selected by the processor.

In an embodiment, a method for determining a respiration rate from a photoplethysmograph signal is provided. The method may include selecting a first portion of the photoplethysmograph signal, mirroring the first portion of the photoplethysmograph signal about a first vertical axis to create a mirrored first portion, selecting a subsequent second portion of the photoplethysmograph signal, mirroring the second portion of the photoplethysmograph signal about a second vertical axis to create a mirrored second portion, combining the mirrored first portion and the mirrored second portion to create a new signal, transforming the new signal into a transformed signal using a wavelet transform, generating a scalogram based at least in part on the transformed signal, identifying a band on the scalogram, extracting ridge information or off-ridge information from the band, transforming the ridge information or the off-ridge information using a wavelet transform into a second transformed signal, generating a second scalogram based at least in part on the second transformed signal, analyzing at least a region of the second scalogram, and determining the respiration rate based on the analysis of the at least a region of the second scalogram.

### BRIEF DESCRIPTION OF THE DRAWINGS

The above and other features of the present disclosure, its nature and various advantages will be more apparent upon consideration of the following detailed description, taken in conjunction with the accompanying drawings in which:

FIG. 1 shows an illustrative pulse oximetry system in accordance with an embodiment;

FIG. 2 is a block diagram of the illustrative pulse oximetry system of FIG. 1 coupled to a patient in accordance with an embodiment;

FIGS. 3(a) and 3(b) show illustrative views of a scalogram derived from a PPG signal in accordance with an embodiment;

FIG. 3(c) shows an illustrative scalogram derived from a signal containing two pertinent components in accordance with an embodiment;

FIG. 3(d) shows an illustrative schematic of signals associated with a ridge in FIG. 3(c) and illustrative schematics of a further wavelet decomposition of these newly derived signals in accordance with an embodiment;

FIGS. 3(e) and 3(f) are flow charts of illustrative steps involved in performing an inverse continuous wavelet transform in accordance with embodiments;

FIG. 4 is a block diagram of an illustrative continuous wavelet processing system in accordance with an embodiment;

FIG. 5 is a flowchart of an illustrative process for selecting and mirroring portions of a signal to create a new signal for further analysis in accordance with an embodiment of the disclosure;

FIG. 6 is a schematic of an illustrative process for reconstructing an up stroke signal and a down stroke signal from an original signal in accordance with an embodiment of the disclosure;

FIG. 7 is a flowchart of an illustrative process for analyzing the reconstructed up stroke signal and down stroke signal of FIG. 6 using secondary wavelet feature decoupling in accordance with an embodiment of the disclosure;

FIG. 8(a) shows a plot of a signal and an illustrative scalogram derived from the signal in accordance with an embodiment of the disclosure;

FIG. 8(b) shows an up stroke signal reconstructed from the signal in FIG. 8(a) and an illustrative scalogram derived from the up stroke signal in accordance with an embodiment of the disclosure;

FIG. 8(c) shows a down stroke signal reconstructed from the signal in FIG. 8(a) and an illustrative scalogram derived from the down stroke signal in accordance with an embodiment of the disclosure; and

FIG. 9 is a flowchart of an illustrative process for applying a sum along amplitudes to a scalogram in accordance with an embodiment of the disclosure.

### DETAILED DESCRIPTION

The present disclosure relates to signal processing and, more particularly, to selecting and mirroring portions of a signal to create a new signal for further analysis. In one exemplary embodiment, the signal may be a PPG signal and the created signal may be further analyzed using continuous wavelet transforms.

In medicine, a plethysmograph is an instrument that measures physiological parameters, such as variations in the size of an organ or body part, through an analysis of the blood passing through or present in the targeted body part, or a depiction of these variations. An oximeter is an instrument that may determine the oxygen saturation of the blood. One common type of oximeter is a pulse oximeter, which determines oxygen saturation by analysis of an optically sensed plethysmograph.

A pulse oximeter is a medical device that may indirectly measure the oxygen saturation of a patients blood (as opposed to measuring oxygen saturation directly by analyzing a blood sample taken from the patient) and changes in blood volume in the skin. Ancillary to the blood oxygen saturation measurement, pulse oximeters may also be used to measure the pulse rate of the patient. Pulse oximeters typically measure and display various blood flow characteristics including, but not limited to, the oxygen saturation of hemoglobin in arterial blood.

An oximeter may include a light sensor that is placed at a site on a patient, typically a fingertip, toe) forehead or earlobe, or in the case of a neonate, across a foot. The oximeter may pass light using a light source through blood perfused tissue and photoelectrically sense the absorption of light in the tissue. For example, the oximeter may measure the intensity of light that is received at the light sensor as a function of time. A signal representing light intensity versus time may be referred to as the photoplethysmogram (PPG) signal. The light intensity or the amount of light absorbed may then be used to calculate the amount of the blood constituent (e.g., oxyhemoglobin) being measured as well as the pulse rate and when each individual pulse occurs.

The light passed through the tissue is selected to be of one or more wavelengths that are absorbed by the blood in an amount representative of the amount of the blood constituent present in the blood. The amount of light passed through the tissue varies in accordance with the changing amount of blood constituent in the tissue and the related light absorption. Red and infrared wavelengths may be used because it has been observed that highly oxygenated blood will absorb relatively less red light and more infrared light than blood with a lower oxygen saturation. By comparing the intensities of two wavelengths at different points in the pulse cycle, it is possible to estimate the blood oxygen saturation of hemoglobin in arterial blood.

When the measured blood parameter is the oxygen saturation of hemoglobin, a convenient starting point assumes a saturation calculation based on Lambeit-Beer's law. The following notation will be used herein:

I(λ,t)=Iₒ(λ)exp(−(sβₒ(λ)+(1−s)βᵣ(λ))l(t)) (1)

where:
λ=wavelength;
t=time;
I=intensity of light detected;
Iₒ=intensity of light transmitted;
s=oxygen saturation;
βₒ, βᵣ=empirically derived absorption coefficients; and
l(t)=a combination of concentration and path length from emitter to detector as a function of time.

The traditional approach measures light absorption at two wavelengths (e.g., red and infrared (IR)), and then calculates saturation by solving for the “ratio of ratios” as follows.

1. First, the natural logarithm of (1) is taken (“log” will be used to represent the natural logarithm) for IR and Red

log I=log Iₒ−(sβₒ+(1−s)βᵣ)l (2)
2. (2) is then differentiated with respect to time

$$\frac{d log I }{d t} = - ( s {β}_{o} + ( 1 - s ) {β}_{r} ) \frac{d l}{d t} \qquad ( 3 )$$

3. Red (3) is divided by IR (3)

$$\frac{d log I ( {λ}_{R} ) / d t}{d log I ( {λ}_{IR } ) / d t} = \frac{s {β}_{o} ( {λ}_{R} ) + ( 1 - s ) {β}_{r} ( {λ}_{R} )}{s {β}_{o} ( {λ}_{IR} ) + ( 1 - s ) {β}_{r} ( {λ}_{IR} )} \qquad ( 4 )$$

### 4. Solving for s

$$s = \frac{\frac{d log I ( {λ}_{IR} )}{d t} {β}_{r} ( {λ}_{R} ) - \frac{d log I ( {λ}_{R} )}{d t} {β}_{r} ( {λ}_{IR} )}{\begin{matrix}\frac{d log I ( {λ}_{R} )}{d t} ( {β}_{o} ( {λ}_{IR} ) - {β}_{r} ( {λ}_{IR} ) ) - \\ \frac{d log I ( {λ}_{IR} )}{d t} ( {β}_{o} ( {λ}_{R} ) - {β}_{r} ( {λ}_{R} ) )\end{matrix}}$$

Note in discrete time

$$\frac{d log I ( λ , t )}{d t} ≃ log I ( λ , {t}_{2} ) - log I ( λ , {t}_{1} )$$

### Using log A-log B=log A/B,

$$\frac{d log I ( λ , t )}{d t} ≃ log ( \frac{I ( {t}_{2} , λ )}{I ( {t}_{1} , λ )} )$$

So, (4) can be rewritten as

$$\frac{\frac{d log I ( {λ}_{R} )}{d t}}{\frac{d log I ( {λ}_{IR} )}{d t}} ≃ \frac{log ( \frac{I ( {t}_{1} , {λ}_{R} )}{I ( {t}_{2} , {λ}_{R} )} )}{log ( \frac{I ( {t}_{1} , {λ}_{IR} )}{I ( {t}_{2} , {λ}_{IR} )} )} = R \qquad ( 5 )$$

where R represents the “ratio of ratios.” Solving (4) for s using (5) gives

$$s = \frac{{β}_{r} ( {λ}_{R} ) - R {β}_{r} ( {λ}_{IR} )}{R ( {β}_{o} ( {λ}_{IR} ) - {β}_{r} ( {λ}_{IR} ) ) - {β}_{o} ( {λ}_{R} ) + {β}_{r} ( {λ}_{R} )} .$$

From (5), R can be calculated using two points (e.g., PPG maximum and minimum), or a family of points. One method using a family of points uses a modified version of (5). Using the relationship

$$\frac{d log I}{d t} = \frac{d I / d t}{I} \qquad ( 6 )$$

now (5) becomes

$$\begin{matrix}\frac{\frac{d log I ( {λ}_{R} )}{d t}}{\frac{d log I ( {λ}_{IR} )}{d t}} ≃ \frac{\frac{I ( {t}_{2} , {λ}_{R} ) - I ( {t}_{1} , {λ}_{R} )}{I ( {t}_{1} , {λ}_{R} )}}{\frac{I ( {t}_{2} , {λ}_{IR} ) - I ( {t}_{1} , {λ}_{IR} )}{I ( {t}_{1} , {λ}_{IR} )}} \\ = \frac{[ I ( {t}_{2} , {λ}_{R} ) - I ( {t}_{1} , {λ}_{R} ) ] I ( {t}_{1} , {λ}_{IR} )}{[ I ( {t}_{2} , {λ}_{IR} ) - I ( {t}_{1} , {λ}_{IR} ) ] I ( {t}_{1} , {λ}_{R} )} \\ = R\end{matrix} \qquad ( 7 )$$

which defines a cluster of points whose slope of y versus x will give R where

x(t)=[I(t₂,λ_{IR})−I(t₁,λ_{IR})]I(t₁,λ_{R})

y(t)=[I(t₂,λ_{R})−I(t₁,λ_{R})]I(t₁,λ_{IR})

y(t)=R×(t) (8)

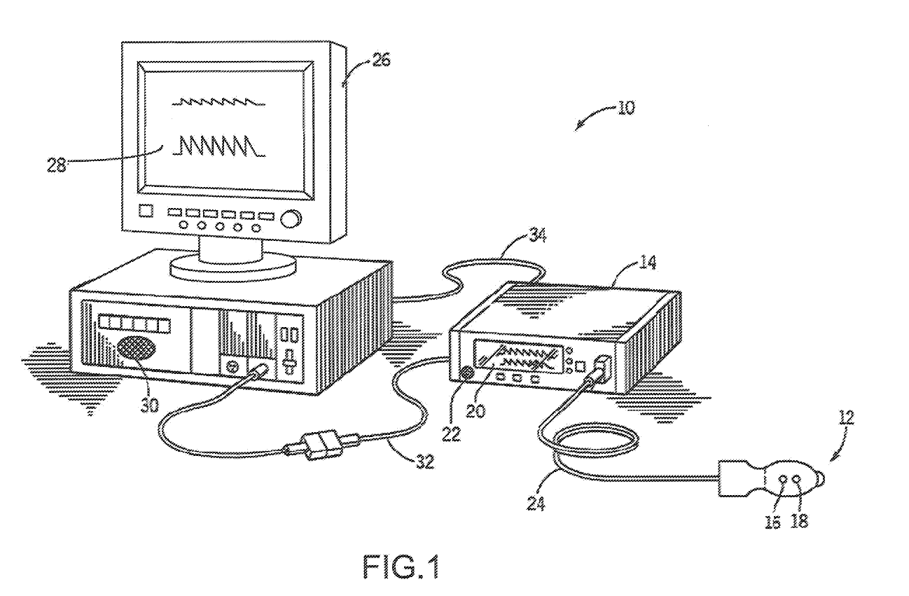

FIG. 1 is a perspective view of an embodiment of a pulse oximetry system 10. System 10 may include a sensor 12 and a pulse oximetry monitor 14. Sensor 12 may include an emitter 16 for emitting light at two or more wavelengths into a patient's tissue. A detector 18 may also be provided in sensor 12 for detecting the light originally from emitter 16 that emanates from the patient's tissue after passing through the tissue.

According to another embodiment and as will be described, system 10 may include a plurality of sensors forming a sensor array in lieu of single sensor 12. Each of the sensors of the sensor array may be a complementary metal oxide semiconductor (CMOS) sensor. Alternatively, each sensor of the array may be charged coupled device (CCD) sensor. In another embodiment, the sensor array may be made up of a combination of CMOS and CCD sensors. The CCD sensor may comprise a photoactive region and a transmission region for receiving and transmitting data whereas the CMOS sensor may be made up of an integrated circuit having an array of pixel sensors. Each pixel may have a photodetector and an active amplifier. According to an embodiment, emitter 16 and detector 18 may be on opposite sides of a digit such as a finger or toe, in which case the light that is emanating from the tissue has passed completely through the digit. In an embodiment, emitter 16 and detector 18 may be arranged so that light from emitter 16 penetrates the tissue and is reflected by the tissue into detector 18, such as a sensor designed to obtain pulse oximetry data from a patient's forehead.

In an embodiment, the sensor or sensor array may be connected to and draw its power from monitor 14 as shown. In another embodiment, the sensor may be wirelessly connected to monitor 14 and include its own battery or similar power supply (not shown). Monitor 14 may be configured to calculate physiological parameters based at least in part on data received from sensor 12 relating to light emission and detection. In an alternative embodiment, the calculations may be performed on the monitoring device itself and the result of the oximetry reading may be passed to monitor 14. Further, monitor 14 may include a display 20 configured to display the physiological parameters or other information about the system. In the embodiment shown, monitor 14 may also include a speaker 22 to provide an audible sound that may be used in various other embodiments, such as for example, sounding an audible alarm in the event that a patient's physiological parameters are not within a predefined normal range.

In an embodiment, sensor 12, or the sensor array, may be communicatively coupled to monitor 14 via a cable 24. However, in other embodiments, a wireless transmission device (not shown) or the like may be used instead of or in addition to cable 24.

In the illustrated embodiment, pulse oximetry system 10 may also include a multi-parameter patient monitor 26. The monitor may be cathode ray tube type, a flat panel display (as shown) such as a liquid crystal display (LCD) or a plasma display, or any other type of monitor now known or later developed. Multi-parameter patient monitor 26 may be configured to calculate physiological parameters and to provide a display 28 for information from monitor 14 and from other medical monitoring devices or systems (not shown). For example, multiparameter patient monitor 26 may be configured to display an estimate of a patient's blood oxygen saturation generated by pulse oximetry monitor 14 (referred to as an “SpO₂” measurement), pulse rate information from monitor 14 and blood pressure from a blood pressure monitor (not shown) on display 28.

Monitor 14 may be communicatively coupled to multi-parameter patient monitor 26 via a cable 32 or 34 that is coupled to a sensor input port or a digital communications port, respectively and/or may communicate wirelessly (not shown). In addition, monitor 14 and/or multi-parameter patient monitor 26 may be coupled to a network to enable the sharing of information with servers or other workstations (not shown). Monitor 14 may be powered by a battery (not shown) or by a conventional power source such as a wall outlet.

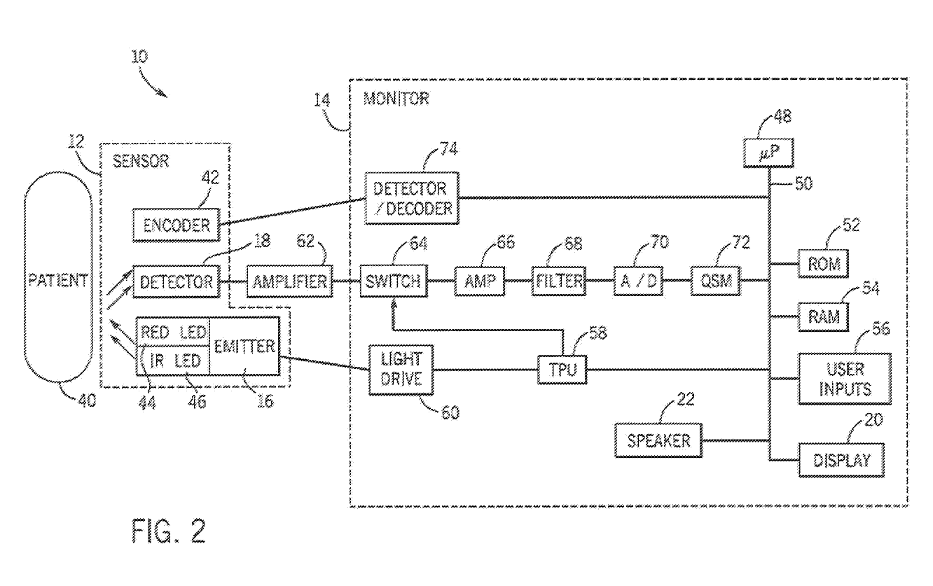

FIG. 2 is a block diagram of a pulse oximetry system, such as pulse oximetry system 10 of FIG. 1, which may be coupled to a patient 40 in accordance with an embodiment. Certain illustrative components of sensor 12 and monitor 14 are illustrated in FIG. 2. Sensor 12 may include emitter 16, detector 18, and encoder 42. In the embodiment shown, emitter 16 may be configured to emit at least two wavelengths of light (e.g., RED and IR) into a patient's tissue 40. Hence, emitter 16 may include a RED light emitting light source such as RED light emitting diode (LED) 44 and an IR light emitting light source such as IR LED 46 for emitting light into the patient's tissue 40 at the wavelengths used to calculate the patient's physiological parameters. In one embodiment, the RED wavelength may be between about 600 nm and about 700 nm, and the IR wavelength may be between about 800 nm and about 1000 nm. In embodiments where a sensor array is used in place of single sensor, each sensor may be configured to emit a single wavelength. For example, a first sensor emits only a RED light while a second only emits an JR light. It will be understood that, as used herein, the term “light” may refer to energy produced by radiative sources and may include one or more of ultrasound, radio, microwave, millimeter wave, infrared, visible, ultraviolet, gamma ray or X-ray electromagnetic radiation. As used herein, light may also include any wavelength within the radio, microwave, infrared, visible, ultraviolet, or X-ray spectra, and that any suitable wavelength of electromagnetic radiation may be appropriate for use with the present techniques. Detector 18 may be chosen to be specifically sensitive to the chosen targeted energy spectrum of the emitter 16.

In an embodiment, detector 18 may be configured to detect the intensity of light at the RED and IR wavelengths. Alternatively, each sensor in the array may be configured to detect an intensity of a single wavelength. In operation, light may enter detector 18 after passing through the patient's tissue 40. Detector 18 may convert the intensity of the received light into an electrical signal. The light intensity is directly related to the absorbance and/or reflectance of light in the tissue 40. That is, when more light at a certain wavelength is absorbed or reflected, less light of that wavelength is received from the tissue by the detector 18. After converting the received light to an electrical signal, detector 18 may send the signal to monitor 14, where physiological parameters may be calculated based on the absorption of the RED and IR wavelengths in the patient's tissue 40. An example of a device configured to perform such calculations is the Model N600x pulse oximeter available from Nellcor Puritan Bennett LLC.

In an embodiment, encoder 42 may contain information about sensor 12, such as what type of sensor it is (e.g., whether the sensor is intended for placement on a forehead or digit) and the wavelengths of light emitted by emitter 16. This information may be used by monitor 14 to select appropriate algorithms, lookup tables and/or calibration coefficients stored in monitor 14 for calculating the patient's physiological parameters.

Encoder 42 may contain information specific to patient 40, such as, for example, the patient's age, weight, and diagnosis. This information may allow monitor 14 to determine, for example, patient-specific threshold ranges in which the patient's physiological parameter measurements should fall and to enable or disable additional physiological parameter algorithms. Encoder 42 may, for instance, be a coded resistor which stores values corresponding to the type of sensor 12 or the type of each sensor in the sensor array, the wavelengths of light emitted by emitter 16 on each sensor of the sensor array, and/or the patient's characteristics. In another embodiment, encoder 42 may include a memory on which one or more of the following information may be stored for communication to monitor 14: the type of the sensor 12; the wavelengths of light emitted by emitter 16; the particular wavelength each sensor in the sensor array is monitoring; a signal threshold for each sensor in the sensor array; any other suitable information; or any combination thereof.

In an embodiment, signals from detector 18 and encoder 42 may be transmitted to monitor 14. In the embodiment shown, monitor 14 may include a general-purpose microprocessor 48 connected to an internal bus 50. Microprocessor 48 may be adapted to execute software, which may include an operating system and one or more applications, as part of performing the functions described herein. Also connected to bus 50 may be a read-only memory (ROM) 52, a random access memory (RAM) 54, user inputs 56, display 20, and speaker 22.

RAM 54 and ROM 52 are Illustrated by way of example, and not limitation. Any suitable computer-readable media may be used in the system for data storage. Computer-readable media are capable of storing information that can be interpreted by microprocessor 48. This information may be data or may take the form of computer-executable instructions, such as software applications, that cause the microprocessor to perform certain functions and/or computer-implemented methods. Depending on the embodiment, such computer-readable media may include computer storage media and communication media. Computer storage media may include volatile and non-volatile, removable and non-removable media implemented in any method or technology for storage of information such as computer-readable instructions, data structures, program modules or other data. Computer storage media may include, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by components of the system.

In the embodiment shown, a time processing unit (TPU) 58 may provide timing control signals to a light drive circuitry 60, which may control when emitter 16 is illuminated and multiplexed timing for the RED LED 44 and the IR LED 46. TPU 58 may also control the gating-in of signals from detector 18 through an amplifier 62 and a switching circuit 64. These signals are sampled at the proper time, depending upon which light source is illuminated. The received signal from detector 18 may be passed through an amplifier 66, a low pass filter 68, and an analog-to-digital converter 70. The digital data may then be stored in a queued serial module (QSM) 72 (or buffer) for later downloading to RAM 54 as QSM 72 fills up. In one embodiment, there may be multiple separate parallel paths having amplifier 66, filter 68, and A/D converter 70 for multiple light wavelengths or spectra received.

In an embodiment, microprocessor 48 may determine the patient's physiological parameters, such as SpO₂ and pulse rate, using various algorithms and/or look-up tables based on the value of the received signals and/or data corresponding to the light received by detector 18. Signals corresponding to information about patient 40, and particularly about the intensity of light emanating from a patient's tissue over time, may be transmitted from encoder 42 to a decoder 74. These signals may include, for example, encoded information relating to patient characteristics. Decoder 74 may translate these signals to enable the microprocessor to determine the thresholds based on algorithms or look-up tables stored in ROM 52. User inputs 56 may be used to enter information about the patient, such as age, weight, height, diagnosis, medications, treatments, and so forth. In an embodiment, display 20 may exhibit a list of values which may generally apply to the patient, such as, for example, age ranges or medication families, which the user may select using user inputs 56.

The optical signal through the tissue can be degraded by noise and motion artifacts, among other sources. One source of noise is ambient light that reaches the light detector. Another source of noise is electromagnetic coupling from other electronic instruments. Movement of the patient also introduces noise and affects the signal. For example, the contact between the detector and the skin, or the emitter and the skin, can be temporarily disrupted when movement causes either to move away from the skin. In addition, because blood is a fluid, it responds differently than the surrounding tissue to inertial effects, thus resulting in momentary changes in volume at the point to which the oximeter probe is attached.

Motion artifact can degrade a pulse oximetry signal relied upon by a physician, without the physician's awareness. This is especially true if the monitoring of the patient is remote, the motion is too small to be observed, or the doctor is watching the instrument or other parts of the patient, and not the sensor site. Processing pulse oximetry (i.e., PPG) signals may involve operations that reduce the amount of noise present in the signals or otherwise identify noise components in order to prevent them from affecting measurements of physiological parameters derived from the PPG signals.

It will be understood that the present disclosure is applicable to any suitable signals and that PPG signals are used merely for illustrative purposes. Those skilled in the art will recognize that the present disclosure has wide applicability to other signals including, but not limited to other biosignals (e.g., electrocardiogram, electroencephalogram, electrogastrogram, electromyogram, heart rate signals, pathological sounds, ultrasound, or any other suitable biosignal), dynamic signals, non-destructive testing signals, condition monitoring signals, fluid signals, geophysical signals, astronomical signals, electrical signals, financial signals including financial indices, sound and speech signals, chemical signals, meteorological signals including climate signals, and/or any other suitable signal, and/or any combination thereof.

In one embodiment, a PPG signal may be transformed using a continuous wavelet transform. Information derived from the transform of the PPG signal (i.e., in wavelet space) may be used to provide measurements of one or more physiological parameters.

The continuous wavelet transform of a signal x(t) in accordance with the present disclosure may be defined as

$$T ( a , b ) = \frac{1}{\sqrt{a}} {\int }_{- ∞}^{+ ∞} x ( t ) {ψ}^{*} ( \frac{t - b}{a} ) d t \qquad ( 9 )$$

where ψ*(t) is the complex conjugate of the wavelet function ψ(t), a is the dilation parameter of the wavelet and b is the location parameter of the wavelet. The transform given by equation (9) may be used to construct a representation of a signal on a transform surface. The transform may be regarded as a time-scale representation. Wavelets are composed of a range of frequencies, one of which may be denoted as the characteristic frequency of the wavelet, where the characteristic frequency associated with the wavelet is inversely proportional to the scale a. One example of a characteristic frequency is the dominant frequency. Each scale of a particular wavelet may have a different characteristic frequency. The underlying mathematical detail required for the implementation within a time-scale can be found, for example, in Paul S. Addison, The Illustrated Wavelet Transform Handbook (Taylor & Francis Group 2002), which is hereby incorporated by reference herein in its entirety.

The continuous wavelet transform decomposes a signal using wavelets, which are generally highly localized in time. The continuous wavelet transform may provide a higher resolution relative to discrete transforms, thus providing the ability to garner more information from signals than typical frequency transforms such as Fourier transforms (or any other spectral techniques) or discrete wavelet transforms. Continuous wavelet transforms allow for the use of multiple wavelets (e.g., on the order of tens, hundreds, thousands, or any other number) that are each scaled in accordance with scales of interest of a signal such that smaller scale components of a signal are transformed using wavelets scaled more compactly than wavelets used to extract larger scale components of the signal. The window size of data to which each wavelet gets applied varies according to scale as well. Thus, a higher resolution transform is possible using continuous wavelets relative to discrete techniques.

In addition, transforms and operations that convert a signal or any other type of data into a spectral (i.e., frequency) domain necessarily create a series of frequency transform values in a two-dimensional coordinate system where the two dimensions may be frequency and, for example, amplitude. For example, any type of Fourier transform would generate such a two-dimensional spectrum. In contrast, wavelet transforms, such as continuous wavelet transforms, are required to be defined in a three-dimensional coordinate system and generate a surface with dimensions of time, scale and, for example, amplitude. Hence, operations performed in a spectral domain cannot be performed in the wavelet domain; instead the wavelet surface must be transformed into a spectrum (i.e., by performing an inverse wavelet transform to convert the wavelet surface into the time domain and then performing a spectral transform from the time domain). Conversely, operations performed in the wavelet domain cannot be performed in the spectral domain; instead a spectrum must first be transformed into a wavelet surface (i.e., by performing an inverse spectral transform to convert the spectral domain into the time domain and then performing a wavelet transform from the time domain). Nor does a cross-section of the three-dimensional wavelet surface along, for example, a particular point in time equate to a frequency spectrum upon which spectral-based techniques may be used. At least because wavelet space includes a time dimension, spectral techniques and wavelet techniques are not interchangeable. It will be understood that converting a system that relies on spectral domain processing to one that relies on wavelet space processing would require significant and fundamental modifications to the system in order to accommodate the wavelet space processing (e.g., to derive a representative energy value for a signal or part of a signal requires integrating twice, across time and scale, in the wavelet domain while, conversely, one integration across frequency is required to derive a representative energy value from a spectral domain). As a further example, to reconstruct a temporal signal requires integrating twice, across time and scale, in the wavelet domain while, conversely, one integration across frequency is required to derive a temporal signal from a spectral domain. It is well known in the art that, in addition to or as an alternative to amplitude, parameters such as energy density, modulus, phase, among others may all be generated using such transforms and that these parameters have distinctly different contexts and meanings when defined in a two-dimensional frequency coordinate system rather than a three-dimensional wavelet coordinate system. For example, the phase of a Fourier system is calculated with respect to a single origin for all frequencies while the phase for a wavelet system is unfolded into two dimensions with respect to a wavelet's location (often in time) and scale.

The energy density function of the wavelet transform, the scalogram, is defined as

S(a,b)=|T(a,b)|² (10)

where ‘∥’ is the modulus operator. The scalogram may be resealed for useful purposes. One common resealing is defined as

$${S}_{R} ( a , b ) = \frac{{\vert T ( a , b ) \vert}^{2}}{a} \qquad ( 11 )$$

and is useful for defining ridges in wavelet space when, for example, the Morlet wavelet is used. Ridges are defined as the locus of points of local maxima in the plane. Any reasonable definition of a ridge may be employed in the method. Also included as a definition of a ridge herein are paths displaced from the locus of the local maxima. A ridge associated with only the locus of points of local maxima in the plane are labeled a “maxima ridge”.

For implementations requiring fast numerical computation, the wavelet transform may be expressed as an approximation using Fourier transforms. Pursuant to the convolution theorem, because the wavelet transform is the cross-correlation of the signal with the wavelet function, the wavelet transform may be approximated in terms of an inverse FFT of the product of the Fourier transform of the signal and the Fourier transform of the wavelet for each required a scale and then multiplying the result by \sqrt{a}.

In the discussion of the technology which follows herein, the “scalogram” may be taken to include all suitable forms of rescaling including, but not limited to, the original unsealed wavelet representation, linear rescaling, any power of the modulus of the wavelet transform, or any other suitable resealing. In addition, for purposes of clarity and conciseness, the term “scalogram” shall be taken to mean the wavelet transform, T(a,b) itself, or any part thereof. For example, the real part of the wavelet transform, the imaginary part of the wavelet transform, the phase of the wavelet transform, any other suitable part of the wavelet transform, or any combination thereof is intended to be conveyed by the term “scalogram”.

A scale, which may be interpreted as a representative temporal period, may be converted to a characteristic frequency of the wavelet function. The characteristic frequency associated with a wavelet of arbitrary a scale is given by

$$f = \frac{{f}_{c}}{a} \qquad ( 12 )$$

where f_{c}, the characteristic frequency of the mother wavelet (i.e., at a=1), becomes a scaling constant and f is the representative or characteristic frequency for the wavelet at arbitrary scale a.

Any suitable wavelet function may be used in connection with the present disclosure. One of the most commonly used complex wavelets, the Morlet wavelet, is defined as:

ω(t)=π^{-1/4}(e^{t2πf}^{₀}^{t}−e^{-(2πf}^{₀}⁾^{²}^{/2})e^{-t}^{²}^{/2} (13)

where f₀ is the central frequency of the mother wavelet. The second term in the parenthesis is known as the correction term, as it corrects for the non-zero mean of the complex sinusoid within the Gaussian window. In practice, it becomes negligible for values of f₀>>0 and can be ignored, in which case, the Morlet wavelet can be written in a simpler form as

$$ψ ( t ) = \frac{1}{{π}^{1 / 4}} {e}^{i 2 π {f}_{0} t} {e}^{- {t}^{2} / 2} \qquad ( 14 )$$

This wavelet is a complex wave within a scaled Gaussian envelope. While both definitions of the Morlet wavelet are included herein, the function of equation (14) is not strictly a wavelet as it has a non-zero mean (i.e. the zero frequency term of its corresponding energy spectrum is non-zero). However, it will be recognized by those skilled in the art that equation (14) may be used in practice with f₀>>0 with minimal error and is included (as well as other similar near wavelet functions) in the definition of a wavelet herein. A more detailed overview of the underlying wavelet theory, including the definition of a wavelet function, can be found in the general literature. Discussed herein is how wavelet transform features may be extracted from the wavelet decomposition of signals. For example, wavelet decomposition of PPG signals may be used to provide clinically useful information within a medical device.

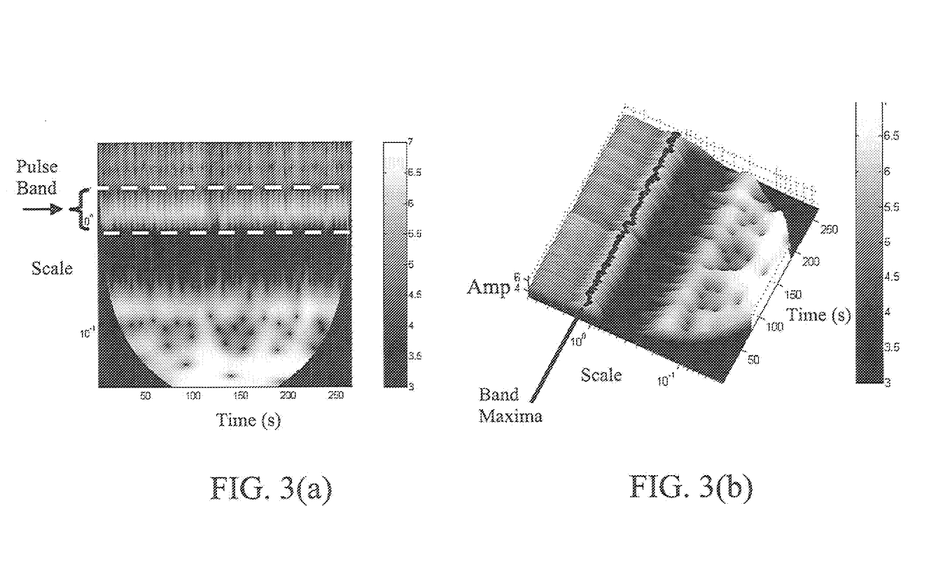

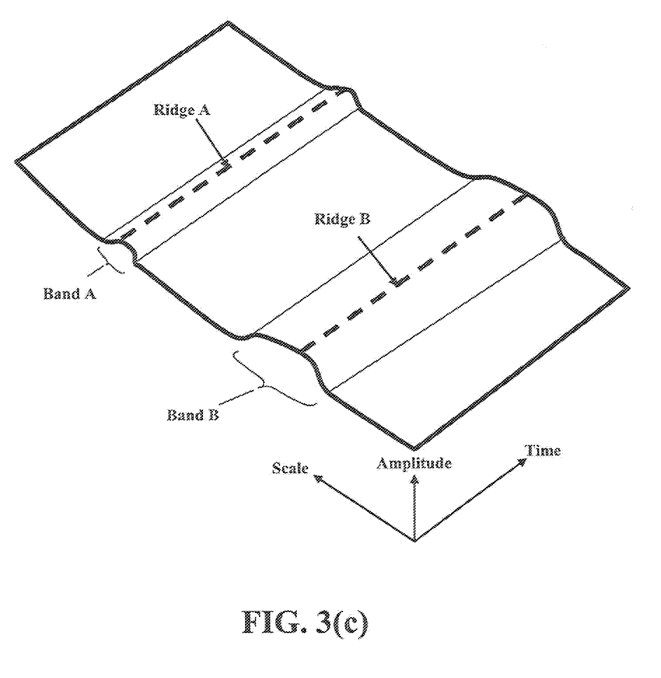

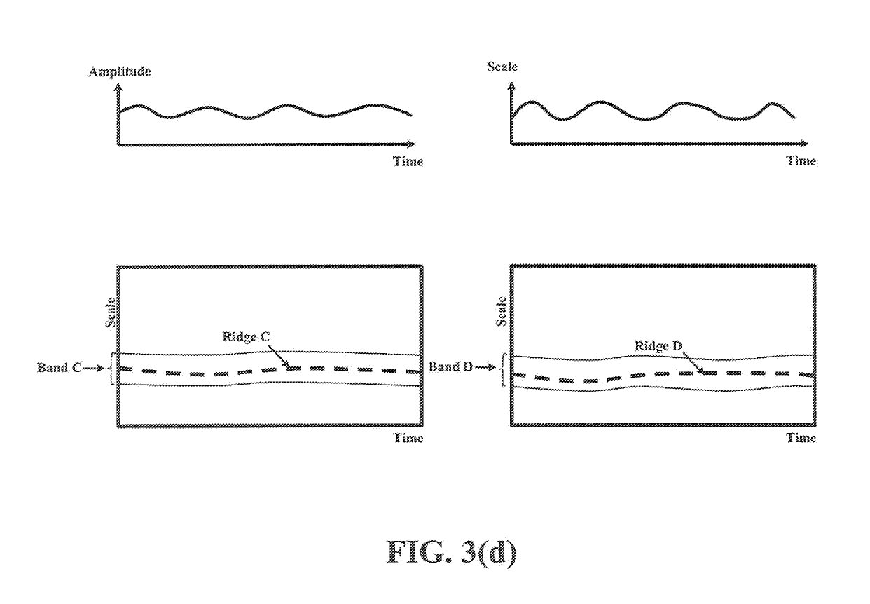

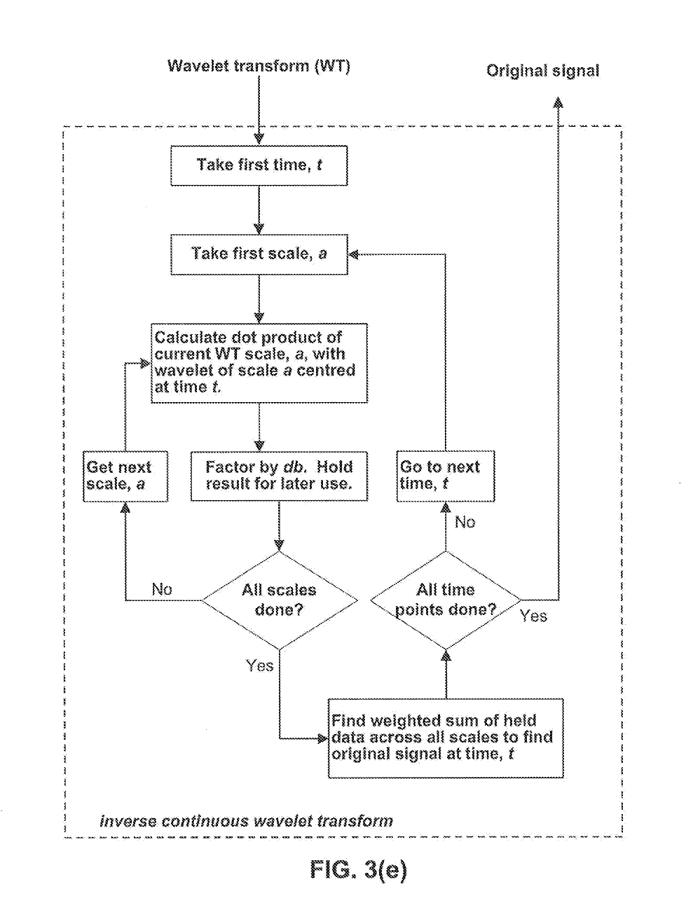

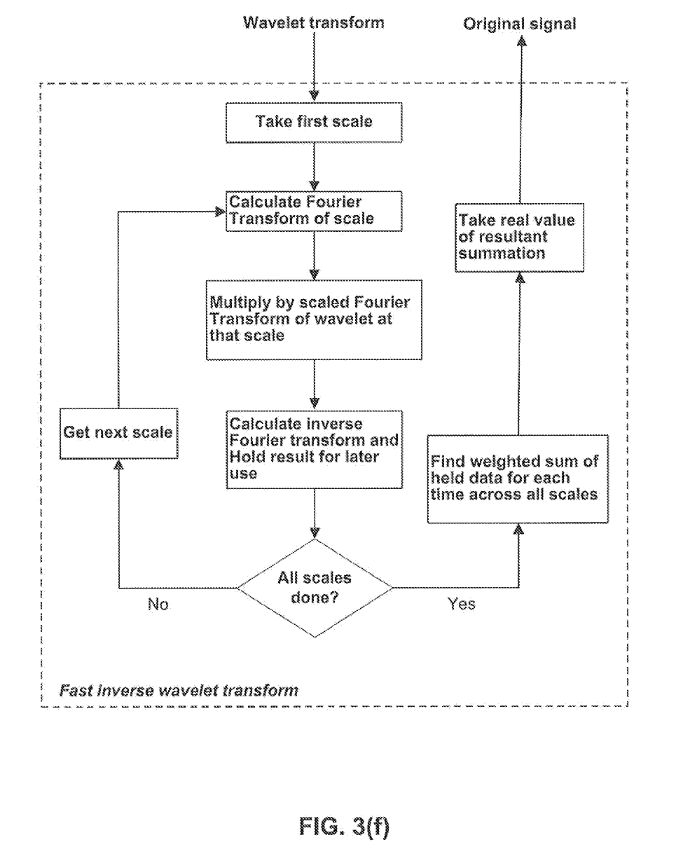

Pertinent repeating features in a signal give rise to a time-scale band in wavelet space or a resealed wavelet space. For example, the pulse component of a PPG signal produces a dominant band in wavelet space at or around the pulse frequency. FIGS. 3(a) and (b) show two views of an illustrative scalogram derived from a PPG signal, according to an embodiment. The figures show an example of the band caused by the pulse component in such a signal. The pulse band is located between the dashed lines in the plot of FIG. 3(a). The band is formed from a series of dominant coalescing features across the scalogram. This can be clearly seen as a raised band across the transform surface in FIG. 3(b) located within the region of scales indicated by the arrow in the plot (corresponding to 60 beats per minute). The maxima of this band with respect to scale is the ridge. The locus of the ridge is shown as a black curve on top of the band in FIG. 3(b). By employing a suitable resealing of the scalogram, such as that given in equation (11), the ridges found in wavelet space may be related to the instantaneous frequency of the signal. In this way, the pulse rate may be obtained from the PPG signal. Instead of rescaling the scalogram, a suitable predefined relationship between the scale obtained from the ridge on the wavelet surface and the actual pulse rate may also be used to determine the pulse rate.

By mapping the time-scale coordinates of the pulse ridge onto the wavelet phase information gained through the wavelet transform, individual pulses may be captured. In this way, both times between individual pulses and the timing of components within each pulse may be monitored and used to detect heart beat anomalies, measure arterial system compliance, or perform any other suitable calculations or diagnostics. Alternative definitions of a ridge may be employed. Alternative relationships between the ridge and the pulse frequency of occurrence may be employed.

As discussed above, pertinent repeating features in the signal give rise to a time-scale band in wavelet space or a rescaled wavelet space. For a periodic signal, this band remains at a constant scale in the time-scale plane. For many real signals, especially biological signals, the band may be non-stationary; varying in scale, amplitude, or both over time. FIG. 3(c) shows an illustrative schematic of a wavelet transform of a signal containing two pertinent components leading to two bands in the transform space, according to an embodiment. These bands are labeled band A and band B on the three-dimensional schematic of the wavelet surface. In this embodiment, the band ridge is defined as the locus of the peak values of these bands with respect to scale. For purposes of discussion, it may be assumed that band B contains the signal information of interest. This will be referred to as the “primary band”. In addition, it may be assumed that the system from which the signal originates, and from which the transform is subsequently derived, exhibits some form of coupling between the signal components in band A and band B. When noise or other erroneous features are present in the signal with similar spectral characteristics of the features of band B then the information within band B can become ambiguous (i.e., obscured, fragmented or missing). In this case, the ridge of band A may be followed in wavelet space and extracted either as an amplitude signal or a scale signal which will be referred to as the “ridge amplitude perturbation” (RAP) signal and the “ridge scale perturbation” (RSP) signal, respectively. The RAP and RSP signals may be extracted by projecting the ridge onto the time-amplitude or time-scale planes, respectively. The top plots of FIG. 3(d) show a schematic of the RAP and RSP signals associated with ridge A in FIG. 3(c). Below these RAP and RSP signals are schematics of a further wavelet decomposition of these newly derived signals. This secondary wavelet decomposition allows for information in the region of band B in FIG. 3(c) to be made available as band C and band D. The ridges of bands C and D may serve as instantaneous time-scale characteristic measures of the signal components causing bands C and D. This technique, which will be referred to herein as secondary wavelet feature decoupling (SWFD), may allow information concerning the nature of the signal components associated with the underlying physical process causing the primary band B (FIG. 3(c)) to be extracted when band B itself is obscured in the presence of noise or other erroneous signal features.

In some instances, an inverse continuous wavelet transform may be desired, such as when modifications to a scalogram (or modifications to the coefficients of a transformed signal) have been made in order to, for example, remove artifacts. In one embodiment, there is an inverse continuous wavelet transform which allows the original signal to be recovered from its wavelet transform by integrating over all scales and locations, a and b:

$$x ( t ) = \frac{1}{{C}_{g}} {\int }_{- ∞}^{∞} {\int }_{0}^{∞} T ( a , b ) \frac{1}{\sqrt{a}} ψ ( \frac{t - b}{a} ) \frac{d a d b}{{a}^{2}} \qquad ( 15 )$$

which may also be written as:

$$x ( t ) = \frac{1}{{C}_{g}} {\int }_{- ∞}^{∞} {\int }_{0}^{∞} T ( a , b ) {ψ}_{a , b} ( t ) \frac{d a d b}{{a}^{2}} \qquad ( 16 )$$

where C_{g} is a scalar value known as the admissibility constant. It is wavelet type dependent and may be calculated from:

$${C}_{g} = {\int }_{0}^{∞} \frac{{\vert \hat{ψ} ( f ) \vert}^{2}}{f} d f \qquad ( 17 )$$

FIG. 3(e) is a flow chart of illustrative steps that may be taken to perform an inverse continuous wavelet transform in accordance with the above discussion. An approximation to the inverse transform may be made by considering equation (15) to be a series of convolutions across scales. It shall be understood that there is no complex conjugate here, unlike for the cross correlations of the forward transform. As well as integrating over all of a and b for each time t, this equation may also take advantage of the convolution theorem which allows the inverse wavelet transform to be executed using a series of multiplications. FIG. 3(f) is a flow chart of illustrative steps that may be taken to perform an approximation of an inverse continuous wavelet transform. It will be understood that any other suitable technique for performing an inverse continuous wavelet transform may be used in accordance with the present disclosure.

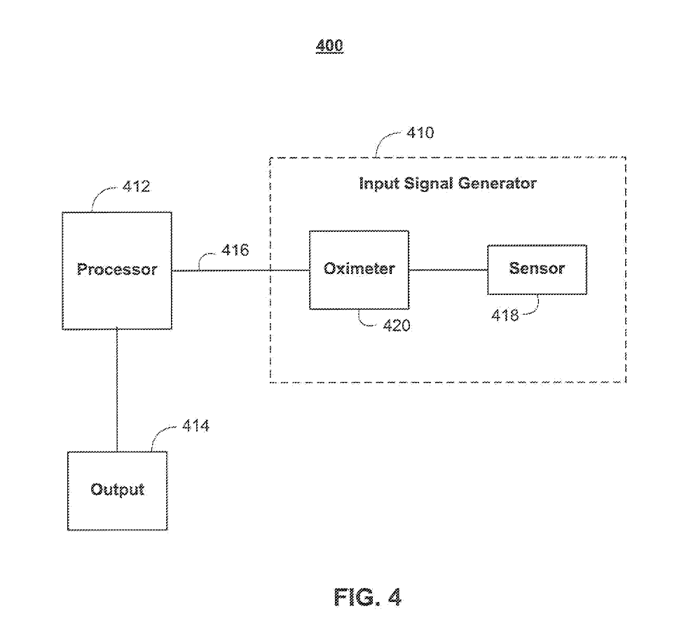

FIG. 4 is an illustrative continuous wavelet processing system 400 in accordance with an embodiment. In this embodiment, input signal generator 410 generates an input signal 416. As illustrated, input signal generator 410 may include oximeter 420 coupled to sensor 418, which may provide as input signal 416, a PPG signal. It will be understood that input signal generator 410 may include any suitable signal source, signal generating data, signal generating equipment, or any combination thereof to produce signal 416. Signal 416 may be any suitable signal or signals, such as, for example, biosignals (e.g., electrocardiogram, electroencephalogram, electrogastrogram, electromyogram, heart rate signals, pathological sounds, ultrasound, or any other suitable biosignal), dynamic signals, non-destructive testing signals, condition monitoring signals, fluid signals, geophysical signals, astronomical signals, electrical signals, financial signals including financial indices, sound and speech signals, chemical signals, meteorological signals including climate signals, and/or any other suitable signal, and/or any combination thereof. In this embodiment, signal 416 may be coupled to processor 412. Processor 412 may be any suitable software, firmware, and/or hardware, and/or combinations thereof for processing signal 416. For example, processor 412 may include one or more hardware processors (e.g., integrated circuits), one or more software modules, computer-readable media such as memory, firmware, or any combination thereof. Processor 412 may, for example, be a computer or may be one or more chips (i.e., integrated circuits). Processor 412 may perform the calculations associated with the continuous wavelet transforms of the present disclosure as well as the calculations associated with any suitable interrogations of the transforms. Processor 412 may perform any suitable signal processing of signal 416 to filter signal 416, such as any suitable band-pass filtering, adaptive filtering, closed-loop filtering, and/or any other suitable filtering, and/or any combination thereof.

Processor 412 may be coupled to one or more memory devices (not shown) or incorporate one or more memory devices such as any suitable volatile memory device (e.g., RAM, registers, etc.), non-volatile memory device (e.g., ROM, EPROM, magnetic storage device, optical storage device, flash memory, etc.), or both. The memory may be used by processor 412 to, for example, store data corresponding to a continuous wavelet transform of input signal 416, such as data representing a scalogram. In one embodiment, data representing a scalogram may be stored in RAM or memory internal to processor 412 as any suitable three-dimensional data structure such as a three-dimensional array that represents the scalogram as energy levels in a time-scale plane. Any other suitable data structure may be used to store data representing a scalogram.

Processor 412 may be coupled to output 414. Output 414 may be any suitable output device such as, for example, one or more medical devices (e.g., a medical monitor that displays various physiological parameters, a medical alarm, or any other suitable medical device that either displays physiological parameters or uses the output of processor 412 as an input), one or more display devices (e.g., monitor, PDA, mobile phone, any other suitable display device, or any combination thereof), one or more audio devices, one or more memory devices (e.g., hard disk drive, flash memory, RAM, optical disk, any other suitable memory device, or any combination thereof), one or more printing devices, any other suitable output device, or any combination thereof.

It will be understood that system 400 may be incorporated into system 10 (FIGS. 1 and 2) in which, for example, input signal generator 410 may be implemented as parts of sensor 12 and monitor 14 and processor 412 may be implemented as part of monitor 14.

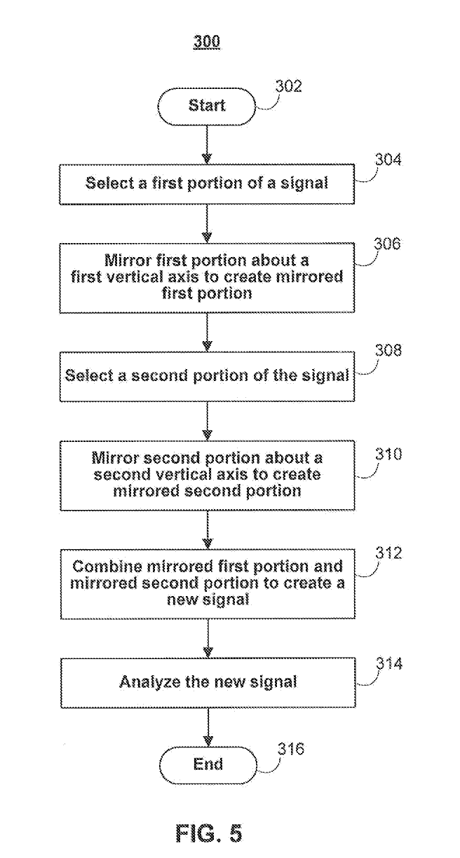

The continuous wavelet processing of the present disclosure will now be discussed in reference to FIGS. 5-9.

FIG. 5 is a flowchart of an illustrative process for selecting and mirroring portions of a signal to create a new signal for further analysis in accordance with an embodiment of the disclosure. Process 300 may begin at step 302. At step 304, a first portion of an original signal may be selected. The original signal may include a signal from any suitable source and may contain one or more repetitive components. For example, the original signal may be a PPG signal. The first portion may be selected using any suitable method based on characteristics of the signal (e.g., using local maximum and minimum values, or using second derivatives to find one or more turning points, of the original signal). The selected portion may correspond to a repetitive portion of the signal. For example, the selected portion may correspond to the up stroke or the down stroke of a PPG signal corresponding to a heartbeat. At step 306, the first portion may be mirrored about any suitable first vertical axis, such as a vertical axis located at the beginning or end of the selected segment, to create a mirrored first portion. By “mirroring,” it is meant that the first portion is reflected about any suitable axis to create a mirrored first portion that contains both the original first portion of the signal and the mirrored, or reflected, portion. By mirroring the first portion about the first vertical axis, a pulse may be created that is symmetrical about the first vertical axis. Process 300 may advance to step 308, in which a second portion may be selected from the original signal. The second portion may be the same as, similar to, or different from the first portion, and may be selected using any suitable method. For example, the second portion may correspond to characteristics of the signal that occur subsequent in time to the first portion. At step 310, the second portion of the original signal may be mirrored about any suitable second vertical axis to create a mirrored second portion. By mirroring the second portion about the second vertical axis, a pulse may be created that is symmetrical about the second vertical axis. In an embodiment, process 300 may advance to step 312, in which the mirrored first portion and the mirrored second portion may be combined to create a new signal. In an embodiment, process 300 may create two new signals: one from the mirrored first portion and one from the mirrored second portion. In this manner, one or more new signals may be created. These new signal may be analyzed further in step 314 using any suitable method. Process 300 may advance to step 316 and end. The foregoing steps of the flowchart are merely illustrative and any suitable modifications may be made. For example, additional portions of the signal may be selected, mirrored (e.g. reflected about any suitable axis to create a pulse that is symmetrical about that axis), and added to the new signal. The process may be performed in real time as the signal is being received or may be performed after a signal has been received. The new signal may be analyzed using a wavelet transform such as a continuous wavelet transform.

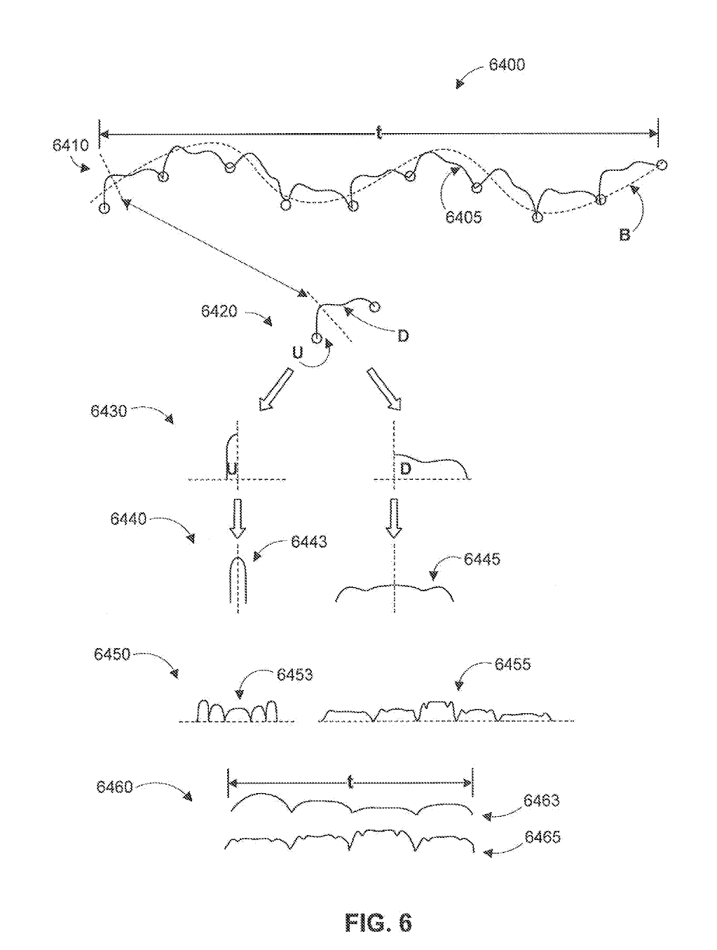

FIG. 6 is a schematic of an illustrative process for reconstructing an up stroke signal and a down stroke signal from an original PPG signal in accordance with an embodiment of the disclosure. Process 6400 may be performed by processor 412 (FIG. 4) or microprocessor 48 (FIG. 2) in real time using a PPG signal obtained by sensor 12 (FIG. 2) or input signal generator 410 (FIG. 4), which may be coupled to patient 40, using a time window smaller than the entire time window over which the PPG signal may be collected. Alternatively, process 6400 may be performed offline on PPG signal samples from QSM 72 (FIG. 2) or from PPG signal samples stored in RAM 54 or ROM 52 (FIG. 2), using the entire time window of data over which the PPG signal was collected.

Process 6400 may begin at step 6410, in which a PPG signal 6405 may be collected by sensor 12 or input signal generator 410 over any suitable time period t to reconstruct an up stroke signal 6463 and/or a down stroke signal 6465. The portion of PPG signal 6405 used to reconstruct up signal 6463 and down signal 6465 may be selected using any suitable approach. For example, the up stroke and the down stroke of PPG signal 6405 may be selected based upon maximum and minimum values of PPG signal 6405. Alternatively, a portion of PPG signal 6405 having an up stroke and a down stroke may be located using second derivatives to find one or more turning points of PPG signal 6405. In an embodiment, processor 412 or microprocessor 48 may include any suitable software, firmware, and/or hardware, and/or combinations thereof for identifying maximum and minimum values of PPG signal 6405 and second derivatives of PPG signal 6405, selecting a portion of PPG signal 6405, and separating one or more up strokes in the portion of PPG signal 6405 from one or more down strokes. The local minimum turning points of PPG signal 6405 are shown in step 6410 using circles. In step 6420, the up stroke and the down stroke may occur between two selected turning points, and the up stroke “U” may be distinguished from the down stroke “D” using a dotted line representing the local maximum value of PPG signal 6405 between and perpendicular to the two turning points of the original baseline B of PPG signal 6405. In one suitable embodiment, the up stroke and the down stroke may be selected after filtering the PPG signal 6405 using, for example, a bandpass filter or low pass filter 68 to filter out frequencies higher and lower than the range of typical heart rates. In another suitable embodiment, the up and down strokes may be detected using techniques described in Watson, U.S. Provisional Application No. 61/077,092, filed Jun. 30, 2008, entitled “Systems and Method for Detecting Pulses,” which is incorporated by reference herein in its entirety. Those skilled in the art will appreciate that any suitable method may be employed for the detection and/or selection of salient portions of the trace including but not limited to pattern matching methods (such as summation of differences or nearest neighbor techniques), syntactic processing methods (such as predicate calculus grammars), and adaptive methods (such as non-monotonic logic inference or artificial neural networks).

In FIG. 6, the original baseline B of PPG signal 6405 is shown as a sinusoidal-like dotted line, according to an embodiment. The baseline B may fluctuate due to the breathing of patient 40, which may cause the PPG signal to oscillate, or twist, in the time plane. For example, PPG signal 6405 may experience amplitude modulation that may be related to dilation of the patients vessels in correspondence with the patient's respiration. PPG signal 6405 may also include a carrier wave that may be based at least in part on the pressure in the patient's venous bed. PPG signal 6405 may also experience frequency modulation that may be based at least in part on a respiratory sinus arrhythmia of the patient. Process 6400 may remove the carrier wave of a PPG signal, the removal of which may be reflected at least in part in the amplitude modulation of the reconstructed up stroke signal and down stroke signal.

Process 6400 may advance to step 6420, in which one up stroke and one down stroke of PPG signal 6405 may be selected by processor 412 or microprocessor 48 using any suitable method. In step 6420, the up stroke and the down stroke may occur between two selected turning points, and the up stroke “U” may be distinguished from the down stroke “D” using a dotted line representing the local maximum value of PPG signal 6405 between and perpendicular to the two turning points. Any other suitable technique may be used to distinguish the up stroke and the down stroke. In an embodiment of the disclosure, up strokes of PPG signal 6405 may be selected for further processing by processor 412 or microprocessor 48 without also selecting down strokes from PPG signal 6405. Similarly, down strokes of PPG signal 6405 may be selected for further processing without also selecting up strokes from PPG signal 6405.

Process 6400 may advance to step 6430, in which the up stroke selected at step 6420 may be separated from the selected down stroke by processor 412 or microprocessor 48 for further processing using any suitable method. For example, the up stroke may be separated from the down stroke at the point where the dotted line, representing the local maximum perpendicular to the two turning points, may intersect the selected portion of PPG signal 6405.

Process 6400 may advance to step 6440, in which each of the selected up stroke “U” and the selected down stroke “D” may be mirrored by processor 412 or microprocessor 48 about any suitable vertical axis. By mirroring each of the selected up stroke “U” and the selected down stroke “D” about a vertical axis, a mirrored up pulse 6443 and a mirrored down pulse 6445 may be created. The mirrored up pulse 6443 and the mirrored down pulse 6445 may each be symmetrical about the respective vertical axis that was used to reflect the selected up stroke “U” and the selected down stroke “D.” The shape of mirrored up pulse 6443 and mirrored down pulse 6445 may depend on which portion of PPG signal 6405 was selected. Because baseline B of PPG signal 6405 may fluctuate, an up stroke and down stroke combination selected from one portion of PPG signal 6405 may have a different amplitude and/or a different frequency than a similar up stroke and down stroke combination from another portion of PPG signal 6405. For example, if a portion of PPG signal 6405 was selected from step 6410 in which the original baseline B was trending downwards, then the up stroke “U” and the resulting mirrored up signal may form a wider, flatter pulse while the down stroke “D” and the resulting mirrored down signal may form a narrower and taller pulse.

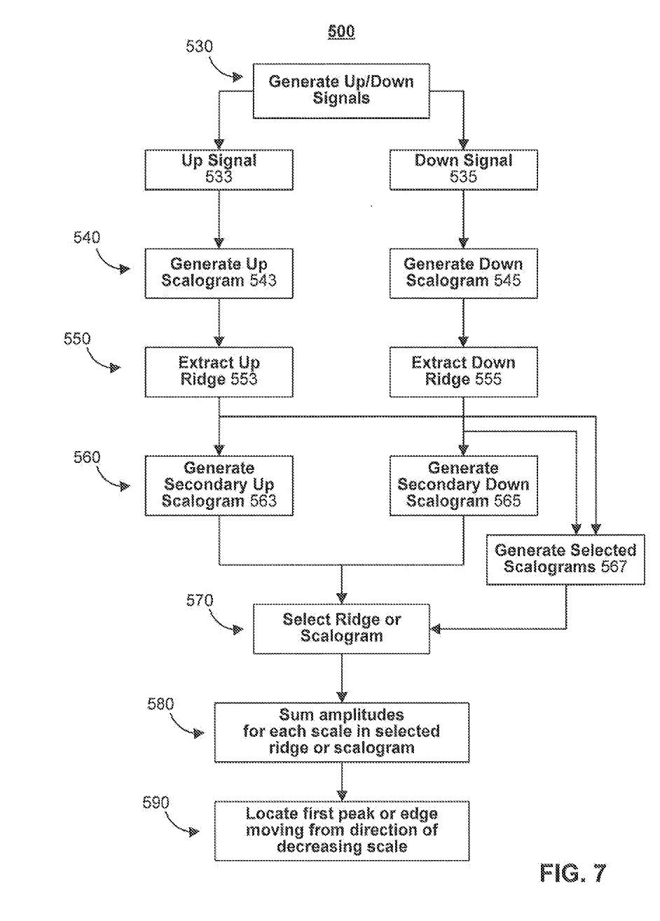

Process 6400 may advance to step 6450, in which each of the mirrored up pulse 6443 and mirrored down pulse 6445 may be added to additional multiple pulses formed from the selection and mirroring of additional up strokes and down strokes from PPG signal 6405 to form mirrored up signal 6453 and mirrored down signal 6455. Alternatively, mirrored up pulse 6443 and mirrored down pulse 6445 may each remain as an individual signal pulse and may be further analyzed by processor 412 or microprocessor 48 as described below with respect to FIG. 7. Each of the pulses in mirrored up signal 6453 and mirrored down signal 6455 may vary in their amplitude and/or their time period, reflecting the amplitude and/or frequency oscillation of PPG signal 6405 in the time plane. Alternatively, each of the mirrored signals could be replicated to form a signal within a desired temporal window instead of forming a signal with a desired number of pulses.

Process 6400 may advance to step 6460, in which each of mirrored up signal 6453 and mirrored down signal 6455 may be further manipulated by processor 412 or microprocessor 48 prior to further analysis, such as by being stretched or compressed to any desired size. Each pulse of the mirrored signals 6453 and 6455 may be expanded or shortened independently of the other pulses in the mirrored signals. For example, each of the pulses in the mirrored signals 6453 and 6455 may be stretched or compressed to make the time period for each pulse equal in size, where all of the time periods together equal the time period t over which PPG signal 6405 was collected or is being analyzed. Alternatively, each pulse of mirrored up signal 6453 and mirrored down signal 6455 may not be stretched to match time period t, but may instead be stretched or compressed to any desired size based at least in part on another time period of PPG signal 6405 or based at least in part on an individual or predetermined number of signal pulses. In an embodiment, each mirrored up pulse may be stretched or compressed to match the size of the up stroke used in the mirroring combined with its corresponding down stroke. The same process may be performed on each mirrored down pulse. In an embodiment, the mirrored pulses in mirrored signals 6453 and 6455 may be equally stretched or compressed to match the time period t over which the PPG signal 6405 was collected or is being analyzed.

The frequency modulation that occurs when one or more of the pulses in mirrored signals 6453 and 6455 is stretched or compressed may be converted into amplitude modulation by processor 412 or microprocessor 48 at step 6460 by increasing or decreasing the amplitude of each of the pulses in the mirrored signals 6453 and 6455 in relation to the amount of individual stretching or compressing described above. This may increase the amplitude modulation that may already exist in the mirrored pulses due to baseline changes in the original PPG signal 6405. Translating the effect of the frequency modulation into amplitude modulation within the mirrored signals 6453 and 6455 may reduce the effect of respiratory sinus arrhythmia of patient 40 on further analysis of PPG signal 6405. The amplitude of the pulses in reconstructed up signal 6463 and/or reconstructed down signal 6465 may be modulated or augmented if each of the pulses was stretched or compressed independently of each other (e.g., to match the time period t over which PPG signal 6405 was collected and to match the period of each other pulse). Alternatively, the amplitude of each of the pulses in reconstructed up signal 6463 or reconstructed down signal 6465 may be the same (not shown) if the frequency modulation applied to the reconstructed signal stretched or compressed each pulse individually to create reconstructed signals with uniform amplitude. In an embodiment, reconstructed up signal 6463 and/or reconstructed down signal 6465 may include pulses that may vary in amplitude and frequency.

In an embodiment of the disclosure, an up stroke, but not a down stroke, may be selected in step 6420, mirrored about a vertical axis in step 6440, replicated in step 6450, and stretched (or compressed) in step 6460. Once the processing (e.g., selecting an up stroke and/or a down stroke, mirroring the strokes, replicating the mirrored pulses, and stretching or compressing the mirrored signals) of mirrored up signal 6453 and mirrored down signal 6455 is completed, then reconstructed up signal 6463 and reconstructed down stroke signal 6465 may be used in further processing by processor 412 or microprocessor 48 as described below with respect to FIG. 7.

FIG. 7 is a flowchart of an illustrative process for analyzing the reconstructed signals of, for example, FIG. 6, using secondary wavelet feature decoupling in accordance with an embodiment of the disclosure. Process 500 may begin at step 530, in which up signal 533 and down signal 535, which may be the same as, and may include some or all of the features of, reconstructed up signal 6463 and reconstructed down signal 6465, respectively, may be generated from any original signal (e.g., a PPG signal) using any suitable method. In an embodiment of the disclosure, only one reconstructed signal (e.g., up signal 533), instead of both reconstructed signals, may be analyzed by process 500.

Process 500 may advance to step 540, in which a primary up scalogram 543 and a primary down scalogram 545 may be derived at least in part from up signal 533 and down signal 535 using any suitable method. For example, up scalogram 543 and down scalogram 545 may be derived using the same method (e.g., using continuous wavelet transforms) that was used to derive the scalograms shown in FIGS. 3(a), 3(b), and 3(c). In an embodiment, processor 412 or microprocessor 48 may perform the calculations associated with the continuous wavelet transforms of up signal 533 and down signal 535. Up scalogram 543 and down scalogram 545 may be derived using a mother wavelet of any suitable characteristic frequency or form such as the Morlet wavelet where fₒ (which is related to its oscillatory nature) may take a value equal to (5.5/2π) Hz, or any other suitable value.

Up scalogram 543 and down scalogram 545 also may be derived over any suitable range of scales. For example, up scalogram 543 and down scalogram 545 may be derived using wavelets within a range of scales whose characteristic frequencies span, for example, approximately 0.8 Hz on either side of the scale corresponding to band A as shown in FIG. 3(c). A narrower range of scales may be used to derive up scalogram 543 and down scalogram 545 to eliminate the inclusion of other artifacts (e.g., noise), to focus on the component of interest within the PPG signal (e.g., the pulse component), and to minimize the number of computations that processor 412 or microprocessor 48 would need to perform. The resultant up scalogram 543 and down scalogram 545 may include ridges corresponding to at least one area of increased energy, such as band A that may be analyzed further using any suitable method, for example using secondary wavelet feature decoupling.

Process 500 may advance to step 550, in which an up ridge 553 and a down ridge 555 may be extracted by processor 412 or microprocessor 48 from up scalogram 543 and down scalogram 545, respectively, using any suitable method. For example, up ridge 553 and down ridge 555 may represent that at a particular scale value, the PPG signal may contain high amplitudes corresponding to the characteristic frequency of that scale. The amplitude and/or scale modulation observed in band A may be the result of the effect of one component of the PPG signal (e.g., a patient's respiration, as shown by breathing band B in FIG. 3(c)) on another component (e.g., a patient's pulse rate, as shown by pulse band A). By extracting and further analyzing up ridge 553 and/or down ridge 555 with respect to band A, information concerning the nature of the signal component associated with the underlying physical process causing the primary band B (FIG. 3(c)) may also be extracted when band B itself is, for example, obscured in the presence of noise or other erroneous signal features.

Process 500 may advance to step 560, in which each of up ridge 553 and down ridge 555 may be transformed further into a secondary up scalogram 563 and a secondary down scalogram 565, respectively, using any suitable method. In an embodiment, processor 412 or microprocessor 48 may perform the calculations associated with any suitable interrogations of the continuous wavelet transforms, including further transforming up ridge 553 and down ridge 555. For example, secondary wavelet feature decoupling may be applied by processor 412 or microprocessor 48 to each of up ridge 553 and down ridge 555 to derive secondary up scalogram 563 and secondary down scalogram 565. The secondary wavelet feature decoupling technique may provide desired information about the primary band B in FIG. 3(c) by examining the amplitude modulation of band A, such amplitude modulation being based at least in part on the presence of the signal component in the PPG signal that may be related to primary band B.

Up ridge 553 or down ridge 555 may be followed in wavelet space and extracted either as an amplitude signal (e.g., the RAP signal as shown in FIG. 3(d)) and/or a scale signal (e.g., the RSP signal as shown in FIG. 3(d)). In an embodiment, an “off-ridge” technique may be employed, in which a path near up ridge 553 or down ridge 555, but not the maxima ridge itself, may be followed in wavelet space. The off-ridge technique may also be used to obtain amplitude modulation in the RAP signal.

The RAP and/or the RSP signal may be extracted by projecting up ridge 553 or down ridge 555 onto the time-amplitude plane. This secondary wavelet decomposition of up ridge 553 and down ridge 555 allows for information concerning the band of interest (e.g., band B in FIG. 3(c)) to be made available as secondary bands (e.g., band C and band D in FIG. 3(d)) for each of secondary up scalogram 563 and secondary down scalogram 565. The ridges of the secondary bands may serve as instantaneous time-scale characteristic measures of the underlying signal components causing the secondary bands, which may be useful in analyzing the signal component associated with the underlying physical process causing the primary band of interest (e.g., the breathing band B) when band B itself may be obscured.

In an embodiment, secondary up scalogram 563 and secondary down scalogram 565 may be derived by processor 412 or microprocessor 48 within a different window of scales than was used to derive up scalogram 543 and down scalogram 545. Secondary up scalogram 563 and secondary down scalogram 565 may be derived using wavelets within a range of scales from any suitable minimum value, such as a scale whose characteristic frequency is approximately 0.07 Hz, up to any suitable maximum value, such as a scale at which the ridge of band A in FIG. 3(c) may be present. For example, using a window between a suitable minimum scale value and a scale value at which band A may be primarily located allows other signal components of the PPG signal (e.g., the breathing band represented by band B) to be analyzed. The window of scale values may still be chosen to eliminate the inclusion of other artifacts (e.g., noise) within the PPG signal.

Secondary up scalogram 563 and secondary down scalogram 565 may be derived by processor 412 or microprocessor 48 using any suitable value for scaling factor f_{c} for the wavelet. For example, the value of f_{c} may be lower than the value of f_{c} used to derive up scalogram 543 and down scalogram 545 to reduce the formation of continuous ridge paths in secondary up scalogram 563 and secondary down scalogram 565. A lower value of f_{c} may decrease the oscillatory nature of a wavelet.

Process 500 may advance to step 567, which may be a repetition of step 560 at a different value of f_{c} The value of f_{c} may be lower than the value used in step 560 so as to break up false ridges within the scalograms of step 567. The ridge fragments formed within the repeated scalograms of step 567 may be used to identify stable regions within secondary up scalogram 563 and secondary down scalogram 565.

Process 500 may advance to step 570, in which the ridge fragments formed within the scalograms of step 560 and the repeated scalograms of step 567 may be analyzed by processor 412 or microprocessor 48 to select one or more desired ridges, using any suitable method. The one or more ridges may be selected, for example, using the techniques described in Watson, et al., U.S. Application No. 61/077,029, filed Jun. 30, 2008, entitled “Systems and Methods for Ridge Selection in Scalograms of Signals,” which is incorporated by reference herein in its entirety. For example, to analyze the ridge fragments, a time window that may vary both in width and in start position (e.g., start time) may be slid across the one or more up repeated scalograms and the one or more down repeated scalogram derived in each of steps 560 and 567. The ridge fragments within the time window may be parameterized in terms of a weighting of the standard deviation of the path that the particular ridge fragment may take, in units of scale, the length of the ridge fragment, the proximity of the ridge fragment to other ridge fragments, and/or any other suitable weighting characteristics. The ridge having the highest weighting may be chosen for further processing by processor 412 or microprocessor 48. In an embodiment, the ridge having the highest weighting may be used to identify and select a stable region within one of the generated scalograms.

Process 500 may advance to step 580, in which a sum along amplitudes technique may be applied by processor 412 or microprocessor 48 to at least a portion of the band corresponding to the selected ridge or at least a portion (e.g., the identified stable region) of the selected secondary scalogram from step 570 using any suitable method. The sum along amplitudes technique may sum, for each scale increment within a range of scales, the amplitude (e.g., the energy) of a selected portion of the secondary scalogram across any suitable time window (e.g., the time window from step 570 with the minimum parameterization value). The resulting sum may thereafter be represented in any suitable manner, such as by plotting the sum for each scale value as a function of scale value. In an embodiment, processor 412 or microprocessor 48 may include any suitable software, firmware, and/or hardware, and/or combinations thereof for generating a sum along amplitudes vector and applying it to the selected secondary scalogram. The technique of applying a sum along amplitudes may be applied to any secondary wavelet feature decoupling method of any suitable original signal. For example, if the ridge fragment selected from step 570 was a ridge fragment of the up repeated scalogram derived in step 567, then the sum along amplitudes technique may be applied to at least a portion of secondary up scalogram 563 containing the selected ridge fragment. Similarly, if the ridge fragment selected from step 570 was a ridge fragment of the down repeated scalogram derived in step 567, then the sum along amplitudes may be applied to at least a portion of secondary down scalogram 565 containing the selected ridge fragment. Alternatively, the sum along amplitudes technique may be applied to the entire secondary up scalogram 563 or secondary down scalogram 565. The sum along amplitudes technique also may be applied to any continuous wavelet transform of any suitable signal, such as a wavelet transform of the original PPG signal 6405 in FIG. 6. In an embodiment, the sum along amplitudes technique may be applied to a scalogram composite, or a superposition formed from the secondary scalograms derived in steps 560 and 567.

Process 500 may then advance to step 590, in which the sum along amplitudes function may be plotted as a function of scale value by processor 412 or microprocessor 48. For example, the amplitude (e.g., energy) of at least a portion of either secondary up scalogram 563 or secondary down scalogram 565 may be summed across time for each scale value increment. In an embodiment, the plot generated at step 590 may be displayed in any suitable manner, including for example, on display 20 (FIG. 2), display 28 (FIG. 2), or output 414 (FIG. 4) for review and analysis by a user of system 10 (FIG. 1) or system 400 (FIG. 4). From the plot, the first peak or edge moving from a direction of decreasing scale along the sum of amplitudes may be identified, either by processor 412 or microprocessor 48 or by a user of system 10 or system 400. The first peak or edge may have analytical value in relation to the original signal from which the secondary wavelet transforms were derived. If secondary up scalogram 563 and secondary down scalogram 565 were derived from a PPG signal, then the first peak or edge of the plot in step 590 may represent the respiration rate of patient 40.

It is to be understood that process 500 may use one or more secondary up scalograms 563, one or more secondary down scalograms 565, or a superposition formed from two or more secondary scalograms, to locate the desired information. Each of up scalogram 543 and down scalogram 545 may be used to generate the amplitude-modulated up ridge 553 and down ridge 555. Up ridge 553 and down ridge 555 may be transformed again, however, before the desired information (e.g., the respiration rate) may be obtained.

Process 500 may be applied to a PPG signal obtained from patient 40 in any suitable manner. In an embodiment, process 500 may take the form of a computer algorithm that may be installed as part of system 10 or system 400. The algorithm may be applied by processor 412 or microprocessor 48 to the PPG signal data in real time as the PPG signal is detected using sensor 12 or using input signal generator 410. In an embodiment, the algorithm may be applied offline to PPG signal samples from QSM 72 or from PPG signal samples stored in RAM 54 or ROM 52. The output of the algorithm, which may be displayed in any suitable manner (e.g., using display 20, display 28, or output 414) may include the respiration rate of patient 40, which may be used by a user of system 10 or system 400 for any suitable purpose (e.g. assessing the respiratory health of patient 40). In an embodiment, the algorithm may provide several benefits in calculating the respiration rate of patient 40. The algorithm uses forced symmetry in that a selected up stroke or down stroke is mirrored about a desired axis to create a pulse that is symmetrical about that desired axis, and a new signal (e.g., up signal 533 or down signal 535) may be created using any suitable number of symmetrical pulses. The forced symmetry may improve the accuracy of the respiration rate determination by permitting a more accurate derivation of scalograms 543 and 545 from up signal 533 and down signal 535, and a more accurate extraction of up ridge 553 and down ridge 555. The process 500 algorithm may also significantly improve the number of samples, or the percentage of patient data, that may be used to determine the patient's respiration rate effectively. Using the process 500 algorithm may further improve the standard deviation of the differences observed between computing the respiration rate using the mirroring algorithm and computing the respiration rate using another method (e.g., by counting one or more respiration features in a patient's nasal thermistor signal). A tradeoff to using the process 500 algorithm, however, may include an increase (e.g., on the order of 7%) in the amount of patient data classified as invalid and therefore unable to be used to determine the respiration rate of patient 40.

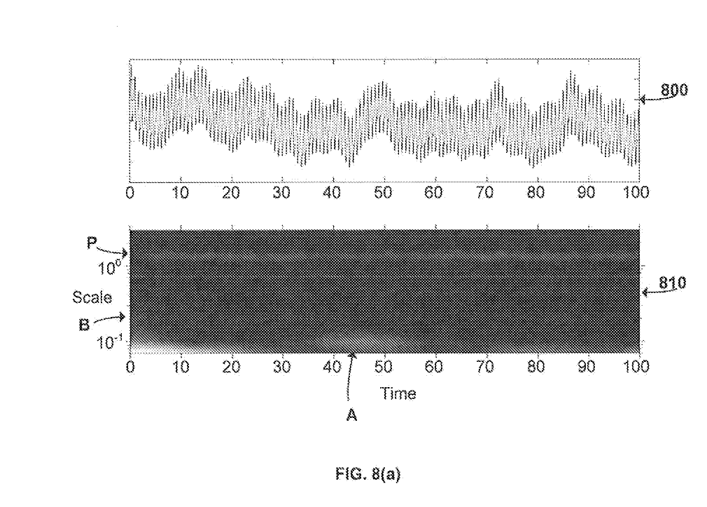

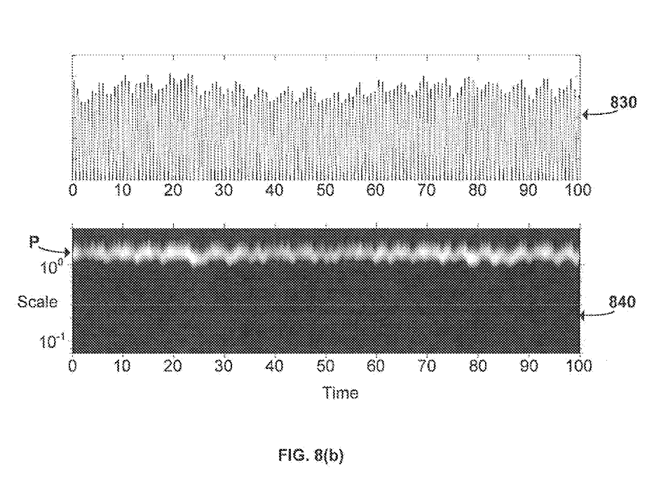

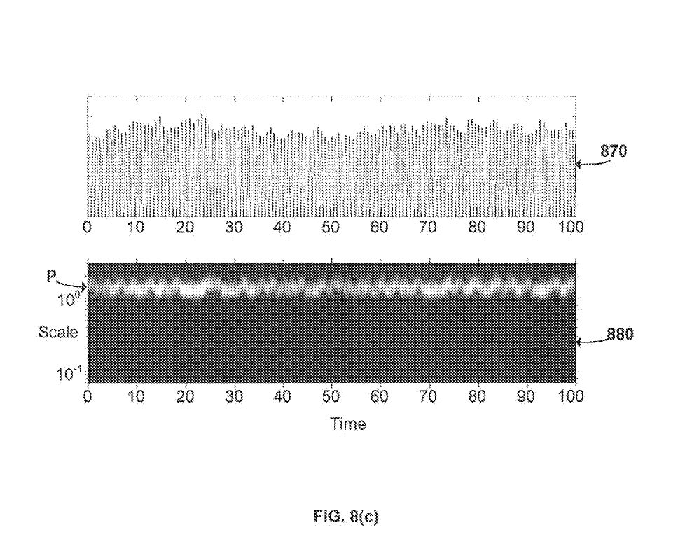

FIG. 8(a) shows a plot of a signal 800, such as a raw absorbance PPG signal, and an illustrative scalogram 810 derived from signal 800 in accordance with an embodiment of the disclosure. Signal 800 may include a red light signal or an infrared light signal obtained from a pulse oximeter sensor attached to a patient as described above. Signal 800 may be plotted as shown in FIG. 8(a) after passing through a portion of the patient's blood perfused tissue (e.g., a fingertip, a toe, a foot). The pulse oximeter sensor may transmit signal 800 to any suitable processing unit (e.g., processor 412 in FIG. 4 or microprocessor 48 in FIG. 2) for further analysis.

Scalogram 810 may be derived from signal 800 by processor 412 or microprocessor 48. Scalogram 810 may include any suitable features, including bands that may relate to clinical parameters of interest, such as a patient's pulse rate (related to pulse band P) or a patient's breathing rate (related to breathing band B), as well as any other artifact that was present in signal 800 (e.g. artifact A). It is to be understood with respect to scalograms 810, 840 (FIG. 8(b)), and 880 (FIG. 8(c)) that the grayscale shown may correspond to high energy components being shaded with a lighter tone and the lower energy components being shaded with a darker tone. The shading may be automatically scaled such that black and white tones may correspond to the lowest and highest energy in each scalogram, respectively. It is to be further understood that the shading used in scalograms 810, 840, and 880 does not necessarily correspond to the same absolute values within each scalogram.

FIG. 8(b) shows an up stroke signal 830 reconstructed from raw absorbance PPG signal 800 and an illustrative scalogram 840 derived from up stroke signal 830 in accordance with an embodiment of the disclosure. Similarly, FIG. 8(c) shows a down stroke signal 870 reconstructed from raw absorbance PPG signal 800 and an illustrative scalogram 880 derived from down stroke signal 870 in accordance with an embodiment of the disclosure. Up stroke signal 830 and down stroke signal 870 may be reconstructed using any suitable methods, including the methods described with respect to FIGS. 5 and 6. Scalogram 840 and scalogram 880 may be derived using any suitable methods, including the method described with respect to FIG. 7. Scalograms 840 and 880 may be derived using a narrower range of scales to eliminate the inclusion of artifacts (e.g., artifact A in scalogram 810), to focus on the component of interest (e.g. pulse band P, which appears lighter in tone and thereby higher in energy in both scalograms 840 and 880 than in scalogram 810), and to minimize the number of computations that processor 412 or microprocessor 48 would need to perform. Scalograms 840 and 880 may include ridges corresponding to at least one area of increased energy, such as band P, that may be analyzed further using any suitable method, for example using secondary wavelet feature decoupling. By extracting and further analyzing the ridges with respect to band P, information concerning the nature of the signal component associated with the underlying physical process causing the breathing band B in scalogram 810 may also be extracted when band B itself is, for example, obscured in the presence of noise or other erroneous signal features.

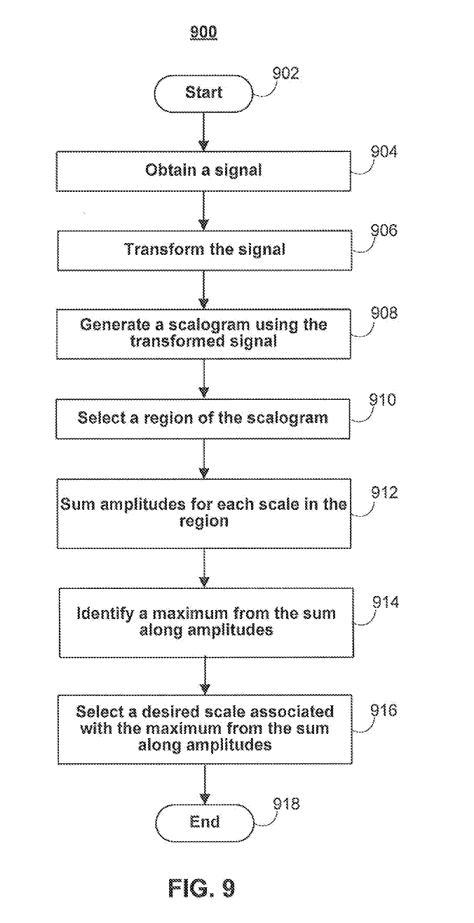

The sum along amplitudes technique, as described in FIG. 7, may be applied to any suitable signal that has been transformed using continuous wavelet transforms. FIG. 9 is a flowchart of an illustrative process for applying a sum along amplitudes to a scalogram in accordance with an embodiment of the disclosure. Process 900 may begin at step 902. At step 904, a signal may be obtained. For example, the signal may include a PPG signal obtained from patient 40 using sensor 12 in system 10 or input signal generator 410 in system 400.

Process 900 may advance to step 906, where the signal may be transformed using any suitable method. For example, the PPG signal may be transformed using a continuous wavelet transform as described above using equation (9). At step 908, a scalogram may be generated in any suitable manner and based at least in part on the transformed signal from step 906. For example, the scalogram may be generated using the energy density function equation (10) and may include some or all of the features described above with respect to FIGS. 3(a), 3(b), and 3(c). In an embodiment, the scalogram of the PPG signal may include any suitable number of bands containing pulse information and respiration information, and each band may include a ridge. The ridge may be continuous or may include any suitable number of ridge fragments.

Process 900 may advance to step 910, where any suitable region of the scalogram may be selected. For example, a portion of the scalogram containing a ridge fragment may be selected. Alternatively, the entire scalogram may be selected. Process 900 may advance to step 912, where, for each scale within the selected region, a sum of the amplitudes (e.g., the energy) across time at that scale may be obtained. Thus, for a region of the scalogram, any suitable number of sums may be calculated within a given time window. The technique may be applied by processor 412 or microprocessor 48 to at least a portion of the band corresponding to the selected ridge or at least a portion of the scalogram. In an embodiment, processor 412 or microprocessor 48 may include any suitable software, firmware, and/or hardware, and/or combinations thereof for generating a sum along amplitudes vector and applying it to the selected region. In an embodiment (not shown), if more than one scalogram was generated at step 908 (e.g., two scalograms may be generated from transforming two PPG signals), then the sum along amplitudes technique may be applied to a scalogram composite, or a superposition formed from the scalograms. In an embodiment (not shown), the sum along amplitudes function (e.g., the sum obtained for each scale value) may be plotted as a function of scale value using any suitable approach. For example, the plot may be generated by processor 412 or microprocessor 48 and may be displayed on display 20 (FIG. 2), display 28 (FIG. 2), or output 414 (FIG. 4) for review and analysis by a user of system 10 (FIG. 1) or system 400 (FIG. 4).

Process 900 may then advance to step 914, where a maximum may be identified (e.g., using processor 412 or microprocessor 48). In an embodiment, the maximum may be identified from a plot by processor 412 or microprocessor 48 or by a user of system 10 or system 400. In an embodiment, the maximum may be the first peak or edge moving from a direction of decreasing scale along the sum of amplitudes. The first peak or edge may have analytical value in relation to a PPG signal from which the scalogram or scalograms may have been generated.

Process 900 may advance to step 916, where a desired scale value that may be associated with the maximum identified in step 914 may be selected. For example, if the original signal was a PPG signal, then the first peak or edge in step 914 may relate to a scale value at that maximum. The scale value may represent the respiration rate of patient 40. Process 900 may then advance to step 918 and end.

It will be understood that the foregoing is only illustrative of the principles of the disclosure, and that the disclosure can be practiced by other than the described embodiments, which are presented for purposes of illustration and not of limitation.

What is claimed is:

1. A signal processing method comprising:
selecting a first portion of an original signal;
mirroring the first portion of the original signal about a first vertical axis to create a mirrored first portion; and
analyzing the mirrored first portion.

2. The method of claim 1, wherein selecting the first portion of the original signal comprises:
identifying a first local minimum value of the original signal;
identifying a subsequent first local maximum value of the original signal; and
selecting the first portion to be the portion of the original signal between the first local minimum value of the original signal and the subsequent first local maximum of the original signal.

3. The method of claim 1, further comprising selecting a subsequent second portion of the original signal;
mirroring the second portion of the original signal about a second vertical axis to create a mirrored second portion; and
analyzing the mirrored second portion.

4. The method of claim 3, wherein selecting the second portion of the original signal comprises:
identifying a second local minimum value of the original signal, wherein the second local minimum value is subsequent to the first local minimum value of the original signal;
identifying a subsequent second local maximum value of the original signal; and
selecting the second portion to be the portion of the original signal between the second local minimum value of the original signal and the subsequent second local maximum of the original signal.

5. The method of claim 3, further comprising combining the mirrored first portion and the mirrored second portion to create a new signal, and analyzing the new signal.

6. The method of claim 5, wherein the first portion of the original signal is selected from a larger first segment of the original signal, wherein the second portion of the original signal is selected from a larger second segment of the original signal, and wherein the first and the second segments are consecutive segments, the method further comprising:
adjusting the length of the new signal by stretching or compressing the new signal to be substantially identical to a combined length of the first and the second segments.

7. The method of claim 5, wherein the first portion of the original signal is selected from a larger first segment of the original signal and wherein the second portion of the original signal is selected from a larger second segment of the original signal, the method further comprising:
adjusting the length of the mirrored first portion by stretching or compressing the mirrored first portion to be a first size; and
adjusting the length of the mirrored second portion by stretching or compressing the mirrored second portion to be a second size.

8. The method of claim 7, further comprising:
adjusting the amplitude of the mirrored first portion based at least in part on the length adjustment of the mirrored first portion; and
adjusting the amplitude of the mirrored second portion based at least in part on the length adjustment of the mirrored second portion.

9. The method of claim 7, wherein the first portion and the second portion of the original signal are selected based at least in part on a second derivative of the original signal.

10. The method of claim 7, wherein analyzing the new signal comprises transforming the new signal into a transformed signal using a wavelet transform.

11. The method of claim 10, further comprising generating a scalogram based at least in part on the transformed signal.

12. The method of claim 11, wherein the original signal is a photoplethysmograph signal from a user, the method further comprising analyzing the scalogram to obtain respiration information of the user.

13. The method of claim 11, wherein the first portion of the original signal and the second portion of the original signal are up strokes of the photoplethysmograph signal.

14. The method of claim 11, wherein the first portion of the original signal and the second portion of the original signal are down strokes of the photoplethysmograph signal.

15. A system for processing a signal, the system comprising:
an input signal generator for generating the signal;
a processor coupled to the input signal generator, wherein the processor is configured to select a first portion of an original signal, mirror the first portion of the original signal about a first vertical axis to create a mirrored first portion, select a subsequent second portion of the original signal, mirror the second portion of the original signal about a second vertical axis to create a mirrored second portion, combine the mirrored first portion and the mirrored second portion to create a new signal, and analyze the new signal; and
an output coupled to the processor, wherein the output is configured to display the new signal analyzed by the processor.

16. The system of claim 15, wherein the input signal generator is a pulse oximeter coupled to a sensor.

17. The system of claim 15, wherein the processor is further configured to adjust an amplitude of the mirrored first portion based at least in part on a length adjustment of the mirrored first portion, and adjust an amplitude of the mirrored second portion based at least in part on a length adjustment of the mirrored second portion.

18. The system of claim 15, wherein analyzing the new signal comprises transforming the new signal into a transformed signal using a wavelet transform.

19. The system of claim 15, wherein the processor is further configured to generate a scalogram based at least in part on the transformed signal.

20. The system of claim 19, wherein the original signal is a photoplethysmograph signal from a user, the processor further configured to analyze the scalogram to obtain respiration information of the user.
